(19) United States
(12) Patent Application Publication
(10) Pub. No.: US 2005/0130886 A1
(43) Pub. Date: Jun. 16, 2005

(54) Methods for promoting antigen presentation and modulating immune responses using cholera toxin and its b subunit

(76) Inventors: Jan Holmgren, Vastra Frolunda (SE); Kristina Eriksson, Molndal (SE)

Correspondence Address: DARBY & DARBY P.C., P. O. BOX 5257, NEW YORK, NY, 10150-5257, US

(21) Appl. No.: 10/478,064

(22) PCT Filed: May 14, 2002
(86) PCT No.: PCT/IB02/01638

Related U.S. Application Data
(60) Provisional application No. 60/290,732, filed on May 14, 2001.

(51) Int. Cl. A61K 38/16

(52) U.S. Cl. 514/12; 424/93.7

(57) ABSTRACT

Use of Cholera toxin and its B subunit as carrier molecules and adjuvants for promoting antigen presentation and increasing the immune response.

This application claims priority under 35 U.S.C. § 119(e) from provisional application Ser. No. 60/290,732, filed May 14, 2001.

### FIELD OF THE INVENTION

This invention relates to the use of Cholera toxin (CT) and its B subunit (CTB) as carrier molecules and adjuvants for promoting antigen presentation and increasing the immune response through their action on different antigen-presenting cells (APC) ex vivo. CTB is an efficient carrier molecule for ex-vivo antigen-pulsing of dendritic cells and other APC, strongly promoting Th2 responses, as evidenced by vaccination of syngeneic mice with the pulsed APC, followed by boosting with the protein or peptide. Treatment of dendritic cells with CT as an adjuvant also markedly enhanced their immunostimulatory capacity, but predisposed also them for eliciting Th1 responses. The apparently different carrier and adjuvant functions of CT and CTB could be combined for maximal potency if the antigen was linked directly to CT prior to DC pulsing, which then predisposed for even stronger Th1 responses and also, in a tumor model system, resulted in development of tumor antigen-specific cytotoxic lymphocytes and ability to eliminate tumor cells even after a solid tumor was established.

### BACKGROUND OF THE INVENTION

Cholera toxin (CT) is a known highly potent mucosal immunogen that additionally has an ability to act as a strong mucosal adjuvant to related as well as unrelated antigens; it also is exceedingly toxic (Abraham et al., J. Immunol. 1992;149:3719-37). The ability of CT to act as an oral adjuvant has been confirmed by a large number of researchers (McGhee, J. R. et al. Vaccine 1992;10 (2): 75-88). Cholera toxin does not fulfill the classical definition of an adjuvant because it stimulates an immune response against itself and its adjuvant activity is closely linked to its immunogenicity (Elson, Fed. Proc. 1987; 46: 1778). The immunomodulating effects of the CT and E. Coli heat-labile enterotoxin (LT) explaining their strong adjuvant activity, include the increase in antigen presentation by several types of B cells, the increase in B cell differentiation to the IgA isotype, the interaction with T cells and the induction of cytokine production (Lintermans, Advanced Drug Delivery Reviews 1995; 18: 73-89).

The less toxic cholera toxin B subunit (CTB) and the closely related E. coli heat-labile enterotoxin B subunit (LTB) are highly efficient carrier molecules for the induction of mucosal antibody responses (Czerkinsky et al., Infect Immun. 1989; 57:1072-7, Holmgren et al., Walter de Gruyter, Berlin, New York 1996; Lipscombe et al., Mol. Microbiol. 1991; 5:1385-1392, McKenzie et al., J. Immunol. 1984; 133:1818-1824) as well as for the induction of mucosally induced systemic T cell (Sun et al., Proc. Natl. Acad. Sci. USA 1994; 91:10795-10799) and B cell oral tolerance (Rask et al., Clin. Exp. Allergy 2000; 30:1024-1032, Tamura et al., Vaccine 1997;15:225-229, Wiedermann et al., Int. Immunol. 1999;11:1717-1724). The later observations have led to the development of antigen-specific tolerogenic strategies to prevent and/or treat T cell mediated autoimmune (4, 43, 48), IgE-mediated allergic (Rask et al., Clin. Exp. Allergy 2000; 30:1024-1032, Tamura et al., Vaccine 1997;15:225-229, Wiedermann et al., Int. Immunol. 1999;11:1717-1724) and infection-induced pathological inflammatory conditions (Sun et al., J. Immunol. 199; 163:1045-1052) by administration of CTB-conjugated antigens through a mucosal surface.

The therapeutic applications of CTB mediated oral tolerance include in animal models the prevention and treatment of T cell-mediated autoimmune diseases (Bergerot et al., Proc Natl Acad Sci USA 1997; 94:4610-4614, Sun et al., Proc. Natl. Acad. Sci. USA 1996 93:7196-7201, Tarkowski et al., Arthritis and Rheumatism 1999; 42:1628-1634), IgE-mediated allergic reactions (Rask et al., Clin. Exp. Allergy 2000; 30:1024-1032, Tamura et al., Vaccine 1997;15:225 229, Wiedermann et al., Int. Immunol. 1999;11:1717-1724) and infection-induced pathological inflammatory conditions (McSorley et al., Eur J Immunol. 1998; 28:424-32, Sun et al., J. Immunol. 1999;163:1045-1052). The mechanism behind CTB's or LTB's efficacy as mucosal carrier molecules has not been fully defined, but is believed to be associated with the strong binding of CTB or LTB to the GMN receptor present on most cells in the body including epithelial cells and leukocytes. Efficient binding to GMI could potentially increase both the uptake of antigen across mucosa and lead to an enhanced presentation of the conjugated molecule to the immune system (Holmgren et al. Walter de Gruyter, Berlin, N.Y. 1996; Proc. natl. acad. Sci. USA. 93:226-230). Another possibility is that CTB has immunodulating properties. Indeed, it has been shown that CTB induces MHC class II expression on B cells (Francis et al., J. Immunol. 1992; 148:1999-2005), enhances antigen presentation by macrophages in the absence of enhanced MHC II expression (Matousek et al., Infect. Immunol. 1998; 66:3480-3484) and blocks the development of diabetes in NOD mice through the development of regulatory cells (Sobel et al., Diabetes 1998; 47:186-191).

Mucosal antigen-specific antibody formation and tolerance induction share the requirement for an initial immune activation (Forster et al. Eur. J. Immunol. 1996; 26:3194-3202). The first step in antigen-specific T cell activation is controlled by antigen-presenting cells (APC) that adsorb, process and present antigens in a complex with MHC class II on the cells surface together with co-stimulatory signals. The responding T cells then are of the CD4+ subset and may develop into either Th1 or Th2 or other helper or regulatory cells. APCcs can also present antigens in a complex with either MHC class I or, more recently described, CD1 molecules on the cell surface. Antigens presented in the context of MHC class I are usually produced intracellularly in the APC, e.g., as a result of an infection with an intracellular bacterial, viral, or parasitic antigen (including recombinant such organisms engineered to express heterologous antigens intracellularly), and are recognized by cognate CD8+ T cells that may develop into CTLs. However, some forms of exogenous antigens, including but not limited to particulate antigen formulations can also be translocated into the APC MHC class I pathway and stimulate CD8+ T cells, including CTLs. The CD1 family of proteins are prominently expressed on most APC and these proteins have recently been found to provide yet another antigen presenting system in addition to that of MHC class I and MHC class II, by presenting predominantly various lipid and glycolipid antigens to CD1 reactive/restricted T cells, including CD4+, CD8+, and CD4/CD8 double negative T cells and cells.

APCs utilize multiple mechanisms for antigen uptake, which vary according to cell type. B cells have membrane-bound antibody receptors that normally recognize and bind one specific antigen whereas other APCs such as dendritic cells (DC) and macrophages (MØ) have a broader range of binding specificities through Fc-receptors and C-type multilectin receptors and can also absorb antigens by macropinocytosis and phagocytosis (Banchereau et al., Nature 1998; 392:245-252). Besides the nature of the antigen, the nature of the APC involved and the cytokines present and/or induced are important determinants for the outcome of the subsequent T cell response. Thus, the level of T cell activation depends on the densities of specific peptide-loaded MHC class II and of co-stimulatory molecules such as CD40, CD80 and CD86 present on the APC surface (McAdam et al., Immunol. Rev. 1998; 165:231-247, van Gool et al. Immunol. Rev. 1996; 153:47-83) as well as on the levels of cytokines produced such as IL-1, IL-12, and IL=18 (Kohno et al. J. Immunol. 1997; 158:1541-1550, O'Garra et al. Immunity. 1998; 8:275-283, Weaver et al., Immunol. Today 1990; 11:49-55).

### Cancer

Cancer remains the second leading cause of death in the United States. There were an estimated 563,100 cancer deaths in 1999. Each year, about 1,222,000 new cancer cases are diagnosed.

There is substantial evidence indicating that the immune system plays a critical role in the prevention of cancer and the control of tumor growth. This includes the occasional observation of spontaneous tumor regression, the correlation of spontaneous regressions with the presence of tumor-infiltrating lymphocytes (TILs) and the identification of TILs that are specific for tumor antigens. However, as evidenced by the incidence rates of cancer, the immune response is often not sufficient to successfully combat the tumor. During the past 40 years, an increased understanding of the immune mechanism has led to the development of approaches to enhance the immune response against the tumor. These have included the expression in tumor cells of genes encoding immunostimulatory molecules and several approaches to enhance tumor antigen presentation. Unfortunately, only limited indications of beneficial effects have been seen and attempts to utilize these new approaches to immunotherapy have been disappointing.

In recent years, there has been a renewed interest in the development of cancer vaccines. This has resulted in part from the identification of new tumor-specific antigens and an increased understanding of the importance of antigen presentation and lymphocyte activation.

The goal of immunotherapy is to augment a patient's immune response to an established tumor. One method of immunotherapy includes the use of adjuvants. Adjuvant substances derived from microorganisms, such as bacillus Calmette-Guerin, heighten the immune response and enhance resistance to tumors in animals. Although bacillus Calmette-Guerin has been tested in many clinical trials, the results have been inconclusive, and the value of this type of bacterial adjuvant therapy remains uncertain (Piessens, W. F., and David, J., “Tumor Immunology”, In: Scientific American Medicine, Vol. 2, Scientific American Books, N.Y., pp. 1-13, 1996).

### SUMMARY OF THE INVENTION

In one embodiment, the invention provides a method for inducing an immune response directed against a non-self, foreign, antigen in a mammal comprising the steps of contacting an antigen presenting cell (APC) with cholera toxin (CT) or its B subunit (CTB) or a related toxin or cell-binding protein, and a foreign antigen(s), incubating the APC/toxin/antigen(s) for a sufficient length of time to activate the APC, washing any unbound APC/toxin/antigen(s), and administering the APCs to a mammal in an effective amount to induce an anti-antigen immune response.

In another aspect the present invention provides a method for inducing an immune response against an antigen in a mammal comprising the steps of contacting antigen presenting cells (APC) ex vivo with cholera toxin or its B subunit or related toxin or cell-binding protein a non-self antigen or group of antigens at a concentration and for a time effective to activate said APCs to promote an immune response directed against said antigen or antigens; washing and removing said unbound cholera toxin or B subunit and non-self antigen or antigens from said APC; and administering to said mammal, an amount of said APCs effective to induce an immune response against said antigen or antigens.

These and other aspects of the present invention will be apparent to those of ordinary skill in the art of the present specification and drawings.

### BRIEF DESCRIPTION OF THE DRAWINGS

FIG. 1(A-B). Construction of the genetic CTB fusion proteins.

(A)Addition of the 17 amino acid OVA peptide to the C terminus of CTB. (B) The insertion of the 13 amino acid HA peptide into CTB molecule replacing residues 56-64 of the mature protein resulting in a protein of 107 instead of 103 amino acids. The first residue of the peptide and residue 55 of CTB are both serine. Additionally, the last residue of the peptide and position 63 of CTB are both lysine. The open blocks show the HA or OVA peptides and the solid blocks represents the mature CTB protein. The serine residue at position 55 and the lysine residue at position 63 of CTB that are shared with the peptide sequence in CTB55-64HA are shown in open boxes.

FIG. 2 (A-C). Conjugation of antigen to CTB enhances antigen presentation in vitro. Spleen cells were incubated with antigen in free form or conjugated to CTB together with purified antigen-specific TCT-transgenic T cells. Data are expressed as the proliferative responses obtained in response to free or CTB-conjugated (A) whole OVA (B) OVA peptide, and (C) HA peptide. (A) APC and OVA-specific TCR transgenic T cells were incubated with 10⁻⁸M OVA, OVA-CTB or 10⁻⁸M OVA+6×10⁻⁹M CTB, (B) APC and OVA-specific TCR transgenic T cells were incubated with graded amounts of OVA peptide (□) or with CTB-conjugated OVA peptide (▪), (C) APC and HA-specific TCR transgenic T cells were incubated with graded amounts of free HA peptide (□), CTB-conjugated HA peptide (●), or the equivalent amounts of un-conjugated HA peptide and CTB (∘).

FIG. 3 (A-D). Secretion of cytokines and chemokines by antigen-pulsed DC. 24 hour cultures of OVA-, CTB-, CT-, OVA-CTB- or OVA-CT-pulsed DC were analysed for levels of IV-12 (A) IV-1β (B), RANTES (C) and MIP-1α (D) by ELISA. Data are expressed as means and standard deviations of at least 2 individual experiments.

### DETAILED DESCRIPTION

All patent applications, patents and literature references cited herein are hereby incorporated by reference in their entirety.

This invention pertains to the unexpected discovery that coupling by chemical conjugation, or resulting from chimeric constructs of Cholera toxin, or its non-toxic B subunit, and a protein or peptide antigen enhance the immune response when exposed to antigen presenting cells.

### Definitions

The terms used in this specification generally have their ordinary meanings in the art, within the context of this invention and in the specific context where each term is used. Certain terms are discussed below, or elsewhere in the specification, to provide additional guidance to the practitioner in describing the compositions and methods of the invention and how to make and use them.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

The terms “mutant” and “mutation” mean any detectable change in genetic material, e.g., DNA, or any process, mechanism or result of such a change. This includes gene mutations, in which the structure (e.g., DNA sequence) of a gene is altered, any gene or DNA arising from any mutation process, and any expression product (e.g., RNA, protein or enzyme) expressed by a modified gene or DNA sequence. The term “variant” may also be used to indicate a modified or altered gene, DNA sequence, RNA, enzyme, cell, etc.; i.e., any kind of mutant.

As used herein, the term “isolated” means that the referenced material is removed from the environment in which it is normally found. Thus, an isolated biological material can be free of cellular components, i.e., components of the cells in which the material is found or produced. In the case of nucleic acid molecules, an isolated nucleic acid includes a PCR product, an isolated mRNA, a cDNA, or a restriction fragment. In another embodiment, an isolated nucleic acid is preferably excised from the chromosome in which it may be found, and more preferably is no longer joined to non-regulatory, non-coding regions, or to other genes, located upstream or downstream of the gene contained by the isolated nucleic acid molecule when found in the chromosome. In yet another embodiment, the isolated nucleic acid lacks one or more introns. Isolated nucleic acid molecules include sequences inserted into plasmids, cosmids, artificial chromosomes, and the like. Thus, in a specific embodiment, a recombinant nucleic acid is an isolated nucleic acid. An isolated protein may be associated with other proteins or nucleic acids, or both, with which it associates in the cell, or with cellular membranes if it is a membrane-associated protein. An isolated organelle, cell, or tissue is removed from the anatomical site in which it is found in an organism. An isolated material may be, but need not be, purified.

The term “purified” as used herein refers to material that has been isolated under conditions that reduce or eliminate the presence of unrelated materials, i.e., contaminants, including native materials from which the material is obtained. For example, a purified protein is preferably substantially free of other proteins or nucleic acids with which it is associated in a cell; a purified nucleic acid molecule is preferably substantially free of proteins or other unrelated nucleic acid molecules with which it can be found within a cell. As used herein, the term “substantially free” is used operationally, in the context of analytical testing of the material. Preferably, purified material substantially free of contaminants is at least 50% pure; more preferably, at least 90% pure, and more preferably still at least 99% pure. Purity can be evaluated by chromatography, gel electrophoresis, immunoassay, composition analysis, biological assay, and other methods known in the art.

Methods for purification are well-known in the art. For example, nucleic acids can be purified by precipitation, chromatography (including preparative solid phase chromatography, oligonucleotide hybridization, and triple helix chromatography), ultracentrifugation, and other means. Polypeptides and proteins can be purified by various methods including, without limitation, preparative disc-gel electrophoresis, isoelectric focusing, HPLC, reversed-phase HPLC, gel filtration, ion exchange and partition chromatography, precipitation and salting-out chromatography, extraction, and countercurrent distribution. For some purposes, it is preferable to produce the polypeptide in a recombinant system in which the protein contains an additional sequence tag that facilitates purification, such as, but not limited to, a polyhistidine sequence, or a sequence that specifically binds to an antibody, such as FLAG and GST. The polypeptide can then be purified from a crude lysate of the host cell by chromatography on an appropriate solid-phase matrix. Alternatively, antibodies produced against the protein or against peptides derived therefrom can be used as purification reagents. Cells can be purified by various techniques, including centrifugation, matrix separation (e.g., nylon wool separation), panning and other immunoselection techniques, depletion (e.g., complement depletion of contaminating cells), and cell sorting (e.g., fluorescence activated cell sorting [FACS]). Other purification methods are possible. A purified material may contain less than about 50%, preferably less than about 75%, and most preferably less than about 90%, of the cellular components with which it was originally associated. The “substantially pure” indicates the highest degree of purity which can be achieved using conventional purification techniques known in the art.

A “coding sequence” or a sequence “encoding” an expression product, such as a RNA, polypeptide, protein or enzyme, is a nucleotide sequence that, when expressed, results in the production of that RNA, polypeptide, protein or enzyme; i.e., the nucleotide sequence “encodes” that RNA or it encodes the amino acid sequence for that polypeptide, protein or enzyme.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently found, for example, by mapping with nuclease S1), as well as protein binding domains (consensus sequences) responsible for the binding of RNA polymerase.

A coding sequence is “under the control of” or is “operatively associated with” transcriptional and translational control sequences in a cell when RNA polymerase transcribes the coding sequence into RNA, which is then trans-RNA spliced (if it contains introns) and, if the sequence encodes a protein, is translated into that protein.

The term “express” and “expression” means allowing or causing the information in a gene or DNA sequence to become manifest, for example producing RNA (such as rRNA or mRNA) or a protein by activating the cellular functions involved in transcription and translation of a corresponding gene or DNA sequence. A DNA sequence is expressed by a cell to form an “expression product” such as an RNA (e.g., a mRNA or a rRNA) or a protein. The expression product itself, e.g., the resulting RNA or protein, may also said to be “expressed” by the cell.

The term “transfection” means the introduction of a foreign nucleic acid into a cell. The term “transformation” means the introduction of a “foreign” (i.e., extrinsic or extracellular) gene, DNA or RNA sequence into a host cell so that the host cell will express the introduced gene or sequence to produce a desired substance, in this invention typically an RNA coded by the introduced gene or sequence, but also a protein or an enzyme coded by the introduced gene or sequence. The introduced gene or sequence may also be called a “cloned” or “foreign” gene or sequence, may include regulatory or control sequences (e.g., start, stop, promoter, signal, secretion or other sequences used by a cell's genetic machinery). The gene or sequence may include nonfunctional sequences or sequences with no known function. A host cell that receives and expresses introduced DNA or RNA has been “transformed” and is a “transformant” or a “clone”. The DNA or RNA introduced to a host cell can come from any source, including cells of the same genus or species as the host cell or cells of a different genus or species.

The terms “vector”, “cloning vector” and “expression vector” mean the vehicle by which a DNA or RNA sequence (e.g., a foreign gene) can be introduced into a host cell so as to transform the host and promote expression (e.g., transcription and translation) of the introduced sequence. Vectors may include plasmids, phages, viruses, etc. and are discussed in greater detail below.

Generally, foreign DNA is inserted at one or more restriction sites of the vector DNA, and then is carried by the vector into a host cell along with the transmissible vector DNA. A segment or sequence of DNA having inserted or added DNA, such as an expression vector, can also be called a “DNA construct.” A common type of vector is a “plasmid”, which generally is a self-contained molecule of double-stranded DNA, usually of bacterial origin, that can readily accept additional (foreign) DNA and which can readily introduced into a suitable host cell. A large number of vectors, including plasmid and fungal vectors, have been described for replication and/or expression in a variety of eukaryotic and prokaryotic hosts. The term “host cell” means any cell of any organism that is selected, modified, transformed, grown or used or manipulated in any way for the production of a substance by the cell. For example, a host cell may be one that is manipulated to express a particular gene, a DNA or RNA sequence, a protein or an enzyme. Host cells can further be used for screening or other assays that are described infra. Host cells may be cultured in vitro or one or more cells in a non-human animal (e.g., a transgenic animal or a transiently transfected animal).

A wide variety of host/expression vector combinations (i.e., expression systems) may be employed in expressing the DNA sequences of this invention. Useful expression vectors, for example, may consist of segments of chromosomal, non-chromosomal and synthetic DNA sequences. Suitable vectors include derivatives of SV40 and known bacterial plasmids, e.g., E. coli plasmids col E1, pCR1, pBR322, pMal-C2, pET, pGEX (Smith et al., Gene 67:31-40, 1988), pMB9 and their derivatives, plasmids such as RP4; phage DNAS, e.g., the numerous derivatives of phage 1, e.g., NM989, and other phage DNA, e.g., M13 and filamentous single stranded phage DNA; yeast plasmids such as the 2 m plasmid or derivatives thereof; vectors useful in eukaryotic cells, such as vectors useful in insect or mammalian cells; vectors derived from combinations of plasmids and phage DNAs, such as plasmids that have been modified to employ phage DNA or other expression control sequences; and the like. In addition, various tumor cells lines can be used in expression systems of the invention.

Yeast expression systems can also be used according to the invention to express any protein of interest. For example, the non-fusion pYES2 vector (XbaI, SphI, ShoI, NotI, GstXI, EcoRI, BstXI, BamH1, SacI, Kpn1, and HindIII cloning sit; Invitrogen) or the fusion pYESHisA, B, C (XbaI, SphI, ShoI, NotI, BstXI, EcoRI, BamH1, SacI, KpnI, and HindIII cloning site, N-terminal peptide purified with ProBond resin and cleaved with enterokinase; Invitrogen), to mention just two, can be employed according to the invention.

Expression of the protein or peptide may be controlled by any promoter/enhancer element known in the art, but these regulatory elements must be functional in the host selected for expression. Promoters which may be used to control gene expression include, but are not limited to, cytomegalovirus (CMV) promoter (U.S. Pat. Nos. 5,385,839 and 5,168,062), the SV40 early promoter region (Benoist and Chambon, 1981, Nature 290:304-310), the promoter contained in the 3′ long terminal repeat of Rous sarcoma virus (Yamamoto, et al., Cell 22:787-797, 1980), the herpes thymidine kinase promoter (Wagner et al., Proc. Natl. Acad. Sci. U.S.A. 78:1441-1445, 1981), the regulatory sequences of the metallothionein gene (Brinster et al., Nature 296:39-42, 1982); prokaryotic expression vectors such as the b-lactamase promoter (Villa-Komaroff, et al., Proc. Natl. Acad. Sci. U.S.A. 75:3727-3731, 1978), or the tac promoter (DeBoer, et al., Proc. Natl. Acad. Sci. U.S.A. 80:21-25, 1983); see also “Useful proteins from recombinant bacteria” in Scientific American, 242:74-94, 1980; promoter elements from yeast or other fungi such as the Gal 4 promoter, the ADC (alcohol dehydrogenase) promoter, PGK (phosphoglycerol kinase) promoter, alkaline phosphatase promoter; and transcriptional control regions that exhibit hematopoietic tissue specificity, in particular: beta-globin gene control region which is active in myeloid cells (Mogram et al., Nature 315:338-340, 1985; Kollias et al., Cell 46:89-94, 1986), hematopoietic stem cell differentiation factor promoters, erythropoietin receptor promoter (Maouche et al., Blood, 15:2557, 1991), etc.

Preferred vectors, particularly for cellular assays in vitro and in vivo, are viral vectors, such as lentiviruses, retroviruses, herpes viruses, adenoviruses, adeno-associated viruses, vaccinia virus, baculovirus, and other recombinant viruses with desirable cellular tropism. Thus, a gene encoding a functional or mutant protein or polypeptide domain fragment thereof can be introduced in vivo, ex vivo, or in vitro using a viral vector or through direct introduction of DNA. Expression in targeted tissues can be effected by targeting the transgenic vector to specific cells, such as with a viral vector or a receptor ligand, or by using a tissue-specific promoter, or both. Targeted gene delivery is described in International Patent Publication WO 95/28494, published October 1995.

Viral vectors commonly used for in vivo or ex vivo targeting and therapy procedures are DNA-based vectors and retroviral vectors. Methods for constructing and using viral vectors are known in the art (see, e.g., Miller and Rosman, BioTechniques, 7:980-990, 1992). Preferably, the viral vectors are replication defective, that is, they are unable to replicate autonomously in the target cell. Preferably, the replication defective virus is a minimal virus, i.e., it retains only the sequences of its genome which are necessary for encapsidating the genome to produce viral particles.

The term “expression system” means a host cell and compatible vector under suitable conditions, e.g., for the expression of a protein coded for by foreign DNA carried by the vector and introduced to the host cell. Common expression systems include E. coli host cells and plasmid vectors, insect host cells such as Sf9, Hi5 or S2 cells and Baculovirus vectors, Drosophila cells (Schneider cells) and expression systems, fish cells and expression systems (including, for example, RTH-149 cells from rainbow trout, which are available from the American Type Culture Collection and have been assigned the accession no. CRL-1710) and mammalian host cells and vectors.

The term “heterologous” refers to a combination of elements not naturally occurring. For example, the present invention includes chimeric RNA molecules that comprise an rRNA sequence and a heterologous RNA sequence which is not part of the rRNA sequence. In this context, the heterologous RNA sequence refers to an RNA sequence that is not naturally located within the ribosomal RNA sequence. Alternatively, the heterologous RNA sequence may be naturally located within the ribosomal RNA sequence, but is found at a location in the rRNA sequence where it does not naturally occur. As another example, heterologous DNA refers to DNA that is not naturally located in the cell, or in a chromosomal site of the cell. Preferably, heterologous DNA includes a gene foreign to the cell. A heterologous expression regulatory element is a regulatory element operatively associated with a different gene that the one it is operatively associated with in nature.

The term “antigen” as used in the invention, is meant to describe a substance that induces a specific immune response when presented to immune cells of an organism. An antigen may thus, in this context, comprise a single immunogenic epitope linked to a carrier protein, or a multiplicity of immunogenic epitopes recognized by a B-cell receptor (ie., antibody on the membrane of the B cell) or a T-cell receptor. A molecule may be both an antigen and an adjuvant (e.g., cholera toxin).

“Antigen presenting cells” (APC) are any mammalian cell which, either constitutively or after stimulation by adjacent cells or their products or by exogenous means, is capable of presenting antigen to cells of the immune system in a manner leading to an immune response. Examples of APC include but are not limited to different types and maturation stages of dendritic cells (DC), niacrophages (MØ), B cells, mast cells, and epithelial cells (the latter exemplified but not limited to epithelial cells derived from skin (keratinocytes), buccal epithelium, intestinal epithelium, or uro-genital tract epithelium).

“In conjunction with” is defined as a formulation between two or more reactants (such as an antigen or part thereof and an immunomodulating agent such as cholera toxin (CT) or cholera toxin B subunit (CTB) or part thereof with or without added cytokines or other agents including immunomodulating nucleic acids or oligonucleotides), wherein these reactants are presented to an APC in conjunction with each other in any effective formulation including but not limited to a mixture, a chemical conjugate, a gene fusion protein or a nucleic acid preparation encoding for either or all of the reactants.

“Cytotoxic T lymphocytes (CTLs)” are CD8+ “killer” T cells that bind epitopes that are part of class I histocompatibility molecules. They secrete molecules that destroy the cell to which they have bound. In addition to ridding the body of cells that have been infected by viruses or transformed by cancer, CTLs are responsible for the rejection of tissue and organ grafts.

“Co-stimulatory molecules” are involved in the interaction between receptor-ligand pairs expressed on the surface of antigen presenting cells and T cells. One exemplary receptor-ligand pair is the B7 co-stimulatory molecules on the surface of DCs and its counter-receptor CD28 or CTLA-4 on T cells (Freeman, et al. (1993) Science 262:909-911; Young, et al. (1992) J. Clin. Invest. 90: 229; Nabavi, et al. Nature 360:266). Other important co-stimulatory molecules are CD40, CD54, CD80, CD86.

The terms “major histocompatibility complex” or “MHC” refers to a complex of genes encoding cell-surface molecules that are required for antigen presentation to T cells and for rapid graft rejection. In humans, the MHC complex is also known as the HLA complex. The proteins encoded by the MHC complex are known as “MHC molecules” and are classified into class I and class II MHC molecules. Class I MHC molecules include membrane heterodimeric proteins made up of an alpha chain encoded in the MHC associated noncovalently with beta-microglobulin. Class I MHC molecules are expressed by nearly all nucleated cells and have been shown to function in antigen presentation to CD8+ T cells. Class I molecules include HLA-A, -B, and -C in humans. Class II MHC molecules also include membrane heterodimeric proteins consisting of non-covalently associated .alpha. and .beta. chains. Class II MHC are known to participate in antigen presentation to CD4+ T cells and, in humans, include HLA-DP, -DQ, and DR.

MHC class II molecules bind to peptides derived from proteins made outside of an antigen presenting cell. In contrast, MHC class I molecules bind to peptides derived from proteins made inside a cell. In order to present peptide in the context of a class II molecule, an APC phagocytosis an antigen into an intracellular vesicle, in which the antigen is cleaved, bound to an MHC class II molecule, and then returned to the surface of the antigen presenting cell.

The term “adjuvant” as used in the invention, is meant to describe a substance added to the formulation to assist in inducing an immune response to the antigen.

The term “therapeutically effective dose” refers to that amount of a compound or compositions that is sufficient to result in a desired activity. Thus, as used to describe a vaccine, a therapeutically effective dose refers to the amount of a compound or compositions (e.g., an antigen) that is sufficient to produce an effective immune response.

Cholera toxin is a bacterial exotoxin from the family of ADP-ribsoylating exotoxins. Most ADP-ribsoylating exotoxins are functionally organized as A:B dimers with a binding B part or subunit and an A part or subunit containing the ADP-ribosyltransferase activity. Such toxins include diphtheria toxin, Pseudomonas exotoxin A, cholera toxin (CT), E. coil heat-labile enterotoxin (LT), pertussis toxin (pertussigen), C. botulinum toxin C2, C. botulinum toxin C3, C. lintosum exoenzyme, B. cereus exoenzyme, Pseudomonas exotoxin S, Staphylococcus aureus EDIN, and B. sphaericus toxin.

Cholera toxin is the archetype example of a ADP-ribsoylating exotoxin that is organized with A and B subunits. The B subunit is the binding subunit and consists of a B-subunit homopentamer which is non-covalently bound to the single-sububnit A subunit. The B-subunit pentamer is arranged in a symmetrical doughnut-shaped structure that binds to GM₁-ganglioside on the target cell. The A subunit serves to ADP ribosylate the alpha subunit of a subset of the hetero trimeric GTP proteins (G proteins) including the Gs protein which results in the elevated intracellular levels of cyclic AMP. This stimulates release of ions and fluid from intestinal cells in the case of cholera.

Cholera toxin and its B subunit (CTB) have adjuvant properties when used as either an intramuscular or oral immunogen (Elson and Dertzbaugh, J Immunol. 1995;154(3):1032-40.; Trach et al., Lancet. 1997;349(9047):231-5.). Another antigen, heat-labile enterotoxin from E. coil (LT) is 80% homologous at the amino acid level with CT and possesses similar binding properties; it also appears to bind the GM₁-ganglioside receptor in the gut and has similar ADP-ribosylating exotoxin activities. Another ADP-ribsoylating exotoxins, Pseudomonas exotoxin A (ETA), binds to the alpha-2-macroglobulin receptor-low density lipoprotein receptor-related protein (Kounnas et al., J Biol Chem. 1993; 268(19):14176-81.). ADP-ribsoylating exotoxins are reviewed by Krueger and Barbieri, Clin Microbiol Rev. 1995; 8(1): 34-47.

CT, CTB and derivatives are defined as any protein which either binds to GM1 ganglioside and/or reacts with polyclonal antiserum to CT, CTB or the A subunit of CT (CTA) as detected by an ELISA or GM1-ELISA test, including but not limited to heat-labile enterotoxin from Escherichia coli (LT), and its composite subunits (LTB and LTA) and to any or all mutated, extended, truncated or otherwise modified forms of CT, LT, its subunits or any other protein that would react with GM1 or with said types of anti-sera as well as any nucleic acid preparation that would encode for a protein that would meet these criteria.

According to the present invention, it has been found CT and CTB promote presentation of coupled antigens not only by DC and B-cells but also by MØ, which are normally poor activators of naive T cells due to their low levels of surface MHC class II. Without wishing to be bound by theory, this enhanced antigen presentation represents an important mechanism contributing to CTB's efficacy as a carrier molecule in vivo.

It has now been unexpectedly discovered that exposure of different APCs to an antigen mixed with, or preferably coupled or fused to CT or CTB by chemical or genetic means can both quantitatively and qualitatively modulate their cognate T cell activating capacity. To this end defined APC populations were pulsed with either free peptide or protein antigens, or with CT or CTB-linked derivatives thereof, or with antigens mixed with CT or CTB. The pulsed APC were then incubated together with purified T cells from antigen-specific TCR transgenic mice and the proliferative responses and cytokine profiles were measured in these cultures. It was found that DC and B cells were efficient APC that could present free peptide and protein antigens to naive transgenic T cells whereas MØ could not. Exposing the different APC to CT or CTB-linked rather than free antigen greatly enhanced their antigen presenting capacity, decreasing by >10⁴-fold the amount of antigen required to stimulate a proliferative response by cognate T cells. Furthermore, MØ also functioned as efficient APC when exposed to the CT or CTB-conjugated antigens. The enhanced T cell proliferative responses obtained were associated with increased levels of secreted IL-12 and IFN-γ, and with increased expression of CD40 and CD86 on the APC surface.

It has been further discovered that DC and other APC from mice, when exposed ex vivo to antigen mixed with or preferably coupled or fused to CT or CTB and then injected in vivo into mice, induced much stronger immune responses than the corresponding APC exposed only to antigen. Further, a differentially modulated immune response was elicited depending on whether CT or CTB was used. This is exemplified below using OVA as the antigen and DC as the APC. Prior to injection into syngeneic mice, bone marrow-derived DC were pulsed with either free OVA, OVA linked to CT or CTB, or mixtures of OVA and CT or CTB. The subsequent immune responses were measured after two DC vaccinations and then again after a subsequent OVA challenge. The results demonstrate that CTB is an efficient carrier molecule for ex vtvo antigen-pulsing of DC, strongly promoting Th2 responses. Treatment of DC with CT adjuvant also markedly enhanced their immunostimulatory capacity but instead predisposed for Th1 responses. The apparently different carrier and adjuvant functions of CT and CTB could be combined for maximal potency if the antigen was linked directly to CT prior to DC pulsing, and then predisposed even stronger for a Th1 response. The Th1 inducing capacity of CT and OVA-CT could be linked to a strong suppression of MIP-1a production by the pulsed DC combined with an upregulation of RANTES production.

It was further discovered that APC-treated ex vivo with a tumor-specific antigen in conjunction with and preferably linked to CT or CTB and then injected into mice suppressed the growth of a transplanted tumor expressing the tumor-specific antigen, and, when coupled to CT, surprisingly induced rejection of an already-established tumor associated with the development of tumor cell-specific CTLs in spleen and/or lymph nodes.

In addition, it was discovered that human APC treated ex vivo with antigen in conjunction with CT or CTB exhibited increased antigen presenting capacity for cognate T cells in a similar fashion as observed in the murine model.

Pursuant to the present invention, APCs are incubated ex vivo with antigen mixed with, conjugated to, or genetically fused to CT, CTB, or a related toxin or cell-binding protein, and thereafter administered to a patient to mount an immune response directed against the antigen. The present invention is directed to an antigen-presenting cell (APC) and any part thereof or derived therefrom, which has been treated in vitro with a specific antigen in conjunction with a bacterial toxin or toxin subunit or other toxin subunit derivative and then administered to a mammalian host. According to the invention, the APC is a dendritic cell, a macrophage, a B lymphocyte, a mast cell or an epithelial cell.

Methods of conjugating proteins or peptides to carrier antigens and generating fusion proteins are well-known in the art. See “Methods” section below for an exemplary description of the generation of antigen-CTB conjugates and fusion proteins. Detailed protocols using CTB are described in Rask et al., Clin. Exp. Allergy 2000, 30:1024-1032 and Schödel et al., Gene 1991; 99:255-59. However,

In a further embodiment, the present invention is directed to an APC and any part thereof which has been treated in vitro with a specific antigen in conjunction with a bacterial toxin or toxin subunit or other toxin subunit derivative wherein the toxin is cholera toxin (CT), the binding subunit of CT (CTB), E. coil LT or LTB, other ADP ribosylating toxins described above and further including but not limited to pertussis toxin from B. periussis, shiga toxin from Shigella and related toxins from either bacteria, or plant toxins such as Abrin and Ricin, or their subunits. Other non-limiting cell-binding toxins or subunits with known immunomodulating properties are also contemplated. Some examples include gram-negative endotoxin such as lipopolysaccharide, tetanus toxin, clostridium difficile toxin, staphylococcal enterotoxins, and streptococcal erythrogenic toxins, and any and all derivatives of these proteins including mutants, extensions or gene fusion proteins. Non-bacterial cell-binding agents include plant lectins (commercially available from Sigma, St. Louis, Mo.) such as Concanavalin A, phytohemagglutinin, and wheat-germ agglutinin, which have mitogenic properties, or replication-defective viral particles and cell attachment proteins (Gilbert Mol. Biotechnol. 2001; 19:169-177, Casal, iotechnol. Genet. Eng. Rev. 2001; 18:73-87)

In another embodiment, fractions or vesicles derived from APC, such as exosomes, can be used in lieu of whole APC. As used herein, exosomes are small (60-100 nanometers), spherical vesicles formed by cells to facilitate intercellular communication. Exosomes produced by dendritic cells (dexosomes) are well characterized, naturally derived products that contain all the known components necessary to activate both adaptive and innate immune responses, Dexosomes, once produced, activate additional immune cells, including other dendritic cells, T-lymphocytes, NK cells and NK T cells. The activation of numerous immune cells is believed to amplify the original signal provided by disease antigens, and an immune response is initiated (Thery et al. J Immunol. 2001; 166:7309-7318, Zitvogel et al. Nature Medicine 1998; 4:594-600).

The methods of the present invention further overcome the problem of the toxicity of the toxins, such as CT, as the unprocessed CT and or CT-linked protein or peptide are removed from the APC preparations prior to administration to a mammal.

The enhanced antigen presentation of CT and CTB-conjugated antigens was shown to be dependent on retained binding activity of the CT- and CTB-antigen complex to GM1 receptors on the APC. The resulting T cell response was shown to be antigen specific. In addition to enhancing antigen presentation by DC and B cells, which express high levels of MHC class II, the conjugation of antigen to CTB also allowed MØ, which express low levels of MHC class II and are normally unable to activate naive T-cells, to present antigen efficiently. Enhanced antigen presentation was associated with an increased expression of IL-12 and IFN-γ, as well as with increased expression of CD40 and CD86, activation and maturation markers, on the APC.

That coupling of antigen to CT or CTB promotes antigen presentation through increased uptake of the coupled antigen through binding to the GM1 receptor on the APC was deduced from experiments showing that both anti-CTB antiserum and free GM1 ganglioside could block the CTB-mediated antigen presentation. CTB has a high affinity for its receptor, the GM1 ganglioside (KA˜1×10⁹ mol⁻¹) (Holmgren et al., J. Exp. Med. 1974;139:801-819). The binding to GM1 results in cellular internalization of CTB into vesicles (Lencer et al., Biochim. Biophys. Acta. 1999;1450:177-190, Rappuoli et al., Immunol. Today. 1999; 20:493-500). Binding to GM1 has previously been shown to be essential for the immunogenicity of CTB (Nashar et al., Proc. Natl. Acad. Sci. USA 1996; 93:226-230) and it has been suggested that GM1 binding represents a danger signal (Rappuoli et al., Immunol. Today 1999; 20:493-500). In the present invention, phenotypic activation of the APC following incubation with CTB-conjugated antigen was evidenced by an up-regulated expression of both CD40 and CD86, as well as a pronounced secretion of IL-12. Thus, without wishing to be bound by theory, it is proposed that GM1 binding greatly improves the uptake of the CTB-conjugated antigen by APC leading to a more abundant presentation of the corresponding peptides on MHC II, which together with the enhanced levels of co-stimulatory molecules such as CD40 and CD86 induced on the APC surface in association with an enhanced IL-12 secretion by the APC augments the T cell activating potential of the APC. It has previously been shown that mannosylation of peptides promotes mannose-receptor mediated antigen absorption by DC, thus reducing the threshold concentration of peptide required by 200-1000-fold (an et al., Eur. J. Immunol. 1997; 27:2426-2435). It is shown herein that CTB-conjugation of antigen represents another potent mechanism for receptor-mediated uptake of antigens by APC, and that this effect is universal as all APC express GM1.

Previous studies have shown that an immune response or tolerance could potentially occur when using CT and CTB. It is clear that whenever CT rather than CTB is used there is never any risk of tolerance induction but an enhancement of the immune response occurs (Elson and Dertzbaugh, Mucosal Immunology 1999, Ed. P. L. Ogra et al., pp. 817-838, Acad. Press; Sun et al., Proc. Natl. Acad. Sci. USA 1996; 93:7196-7201). Otherwise, it is mainly a question of the type of antigen, immunogen versus tolerogen. Surprisingly, when CTB-linked antigens are used for mucosal administration ex vivo, tolerance is induced not only to tolerogens but also to weak immunogens such as ovalbumin. In contrast, when CTB-linked antigens (including OVA) given to dendritic cells as desribed herein (or the other APC) for in vitro vaccination followed by in vivo infusion, enhancement of the immune response and never any evidence of tolerance is consistently observed. For the bone marrow derived or human blood monocyte derived dendritic cells obtained by tissue culture, this probably reflects the fact that these DC are already rather mature (it is generally considered that mature DC give rise to immunity, whereas immature DC can induce tolerance). Both CT and CTB induce expression of co-stimulatory molecules such as CD40 and CD86 on DC and other APC and (probably most importantly) induce expression of IL-1 as well. Detection of the latter cytokine could be used as an in vitro test assurance that there will be an immune response and no risk of tolerance induction.

Pursuant to the present invention, the immunogenic proteins and peptides, preferably chemically coupled to or genetically fused with CTB or CT, or, alternatively, mixed with these proteins (preferably with CT), are incubated with the APC, which are then washed and administered to mammals in amounts effective to mount an immune response against the proteins or peptides.

In one embodiment, the APC is isolated or generated from the same mammalian host as the one to whom the APC, either intact or as a sub-cellular form, will be administered.

In a further embodiment, the APC is isolated or generated from another mammalian host than the one to whom the APC, either intact or as a sub-cellular form, will be administered.

In this preferred embodiment APCs are isolated and treated in vitro (incubated for between about 1 hour and about 24 hours) with immunogenic peptides chemically coupled or genetically fused with CT or CTB or are mixed with CT or CTB and administered to mammals in order to induce an immune response. In this embodiment, amounts of APC effective to induce an immune response are employed. APCs can be obtained from the recipient or from a donor.

The isolation of human APCs, DC, B cells and M\acute{Ø} is described below in the “Methods” section. DC are particularly preferred for use in the present invention.

In a particularly preferred embodiment, the peptides or proteins are chemically coupled or genetically fused to CT. Methods for chemically coupling peptides and proteins to CT and CTB are set forth in the “Methods” section below. Any method of chemical coupling may be used. Preferably the coupling uses commercially available functional adaptors. These are all commercially available from numerous sources such as Sigma Chemical Co. (St. Louis, Mo.) and Merck (White House Station, N.J.). Peptide-protein carrier polymers may be formed using conventional crosslinking agents such as carbodiimides. Examples of carbodiimides are 1-cyclohexyl-3-(2-morpholinyl-(4-ethyl) carbodiimide (CMC), 1-ethyl-3-(3-dimethyaminopropyl) carbodiimide (EDC) and 1-ethyl-3-(4-azonia-44-dimethylpentyl) carbodiimide.

Examples of other suitable crosslinking agents are cyanogen bromide, glutaraldehyde and succinic anhydride. In general, any of a number of homobifunctional agents including a homobifunctional aldehyde, a homobifunctional epoxide, a homobifunctional imidoester, a homobifunctional N-hydroxysuccinimide ester, a homobifunctional maleirnide, a homobifunctional alkyl halide, a homobifunctional pyridyl disulfide, a homobifunctional aryl halide, a homobifunctional hydrazide, a homobifunctional diazonium derivative and a homobifunctional photoreactive compound may be used. Also included are heterobifunctional compounds, for example, compounds having an amine-reactive and a sulfhydryl-reactive group, compounds with an amine-reactive and a photoreactive group and compounds with a carbonyl-reactive and a sulfhydryl-reactive group.

Specific examples of such homobifunctional crosslinking agents include the bifunctional N-hydroxysuccinimide esters dithiobis(succinimidylpropionate), disuccinimidyl suberate, and disuccinimidyl tartarate; the bifunctional imidoesters dimethyl adipimidate, dimethyl pimelimidate, and dimethyl suberimidate; the bifunctional sulfhydryl-reactive crosslinkers 1,4-di-[3′-(2′-pyridyldithio) propion-amido]butane, bismaleimidohexane, and bis-N-maleimido-1,8-octane; the bifunctional aryl halides 1,5-difluoro-2,4-dinitrobenzene and 4,4′-difluoro-3,3′-dinitrophenylsulfone; bifunctional photoreactive agents such as bis-[b-4-azidosalicylamide)ethyl]disulfide; the bifunctional aldehydes formaldehyde, malondialdehyde, succinaldehyde, glutaraldehyde, and adiphaldehyde; a bifunctional epoxied such as 1,4-butaneodiol diglycidyl ether, the bifunctional hydrazides adipic acid dihydrazide, carbohydrazide, and succinic acid dihydrazide; the bifunctional diazoniums o-tolidine, diazotized and bis-diazotized benzidine; the bifunctional alkylhalides N1N′-ethylene-bis(iodoacetamide), N1N′-hexamethylene-bis(iodoacetamide), N1N′-undecamethylene-bis(iodoacetamide), as well as benzylhalides and halomustards, such as a1a′-diiodo-p-xylene sulfonic acid and tri)2-chloroethyl)amine, respectively.

Examples of other common heterobifunctional crosslinking agents that may be used to effect the conjugation of proteins to peptides include, but are not limited to, SMCC succinimidyl-4-(N-maleimidomethyl)cyclohexane-1-carboxylate), MBS (m-maleimidobenzoyl-N-hydroxysuccinimide ester), SIAB (N-succinimidyl(4-iodacteyl) aminobenzoate), SMPB (succinimidyl-4-(p-maleimidophenyl)butyrate), GMBS (N-(γ-maleimidobutyryloxy)succinimide ester), MPHB (4-(4-N-maleimidopohenyl) butyric acid hydrazide), M2C2H (4-(N-maleimidomethyl) cyclohexane-1-carboxyl-hydrazide), SMPT (succinimidyloxycarbonyl-á-methyl-á-(2-pyridyldithio)toluene), and SPDP (N-succinimidyl 3-(2-pyridyldithio) propionate).

Crosslinking may be accomplished by coupling a carbonyl group to an amine group or to a hydrazide group by reductive amination.

In addition, methods of generating recombinant polypetides are described in e.g., Sambrook, Fritsch & Maniatis, Molecular Cloning: A Laboratory Manual, Second Edition (1989) Cold Spring Harbor Laboratory Press, Cold Spring Harbor, N.Y.; DNA Cloning: A Practical Approach, Volumes I and II (D. N. Glover ed. 1985); Oligonucleotide Synthesis (M. J. Gait ed. 1984); Nucleic Acid Hybridization [B. D. Hames & S. J. ÊHiggins eds. (1985)]; Transcription And Translation [B. D. Hames & S. J. Higgins, eds. (1984)]; Animal Cell Culture [R. I. Freshney, ed. (1986)]; Immobilized Cells And Enzymes [IRL Press, (1986)]; B. ÊPerbal, A Practical Guide To Molecular Cloning (1984); F. M. Ausubel et al. (eds.), Current Protocols in Molecular Biology, John Wiley & Sons, Inc. (1994).

Genetic fusion of immunogenetic peptides with CTB is shown below in the Methods section. Amounts of APC effective to induce an immnune response can be calculated as follows. Standard clinical protocols using in vitro-antigen-treated APC for example, for vaccination in humans are employed. 1-10 million cells per infusion dose are initially employed and the number of doses may be repeated 5-10 times or more. The effective amount are determined by typical phase I experimentation in humans by starting with a relatively low number of cells, e.g., 10,000 cells and treating with three different amounts of antigen in conjunction with CT (or CTB) given en two occasions within a two week interval. Patients are monitored to ensure that there are no severe adverse reactions. The immune response (antigen-specific antibodies plus antigen-specific T-cell proliferation) is also measured. If the treatment is regarded as being acceptable from a safety point, 100,000 cells treated in the same way are used; then 1,000,000 cells are used. The number of cells and the dose of antigen for expanded clinical trials may be determined from these studies. Although one would expect that only MHC-syngeneic cells would work, the present inventors have determined that heterologous APC can be employed in the present invention. Without wishing to be bound by theory, it is believed that some material from the initially (ex vivo) treated APC is captured by other APC in the recipient in vivo for triggering the immune response. It is believed that a small immune response reaction to the foreign APC in vivo may indeed help the immune response to the carried antigen by providing cytokine inflammatory stimulation.

The amounts of antigen effective to induce an immune response can be determined experimentally using routines procedures well-known to those of ordinary skill in the art. Thus, a relatively low dose, e.g., 1 microgram per 10⁶ can initially be used and the APC monitored for the induction of cell associated antigens (CD 40, CD 80 or CD 86) and/or the release of cytokines (IFN-gama, TMF etc.). The amount can be adjusted so that the maximal activation is achieved.

In this embodiment of the present invention, there is no danger that toxic amounts of cholera toxin will be infused into patients because the APC are extensively washed before they are administered into the recipient. Any physiological saline or other cell physiological buffers or tissue culture medium, e.g., Iscove's medium or Hank's Buffered Salt Solution (Gibco, Rockville, Md.) may be used for treating the APC's ex vivo and for washing and infusing the cells to patients.

Pursuant to the present invention, an effective amount of antigen would broadly range between about 0.01 and 10 μg. Effective amounts of purified antigen for use with CTB would range between about 0.1 and 1 μg per 10³-10⁶ APC. For CT effective amounts would range between about 0.001 μg and 0.1 μg. An effective amount of tumor cell extract would range between about 0.1 and 1 μg.

The APCs of the present invention may be administered parenterally, including intravenous (i.v.), intramuscular (i.m.), subcutaneous (s.c.), intraperitoneally (i.p.) and intracolonically (i.c.). i.v., s.c. and i.m. are preferred for humans because they have proven useful experimentally.

APC's, either before or after activation, can immediately be used (infused into a mammal) or stored until needed. For storage prior to use, isolated APC can be frozen up to several weeks, or placed in methylcellulose gel for several days.

In another embodiment, mutated CT or CTB or other toxins are used. Examples of such mutations include any point mutation, deletion or insertion into these toxins, subunits or other proteins as well as any peptide extensions to these proteins whether placed in the amino-end, the carboxy-end or elsewhere in the protein and irrespective of whether these peptides have immunological properties by being B cell epitopes, T cell epitopes or otherwise stimulating or deviating the immune response. For example, a number of such mutants have been described in the literature (Backström et al; Gene 1995; 165: 163-171; Backström et al., Gene 1996; 169: 211-217; Schödel et al., Gene 1991; 99: 255-259; Dertzbaugh et al., Infect. Immun. 1990; 58: 70-79).

CT and CTB are commercially available from List Biological Laboratories, Inc. Campbell, Callip 95008, USA. Recombinant (rCTB) SBL is commercially available from Vaccin, Stockholm, Sweden. E. coli LT and LTB may be obtained as described by Rask et al. APMIS 2000; 108:178-86.

Antigen Presenting Cells

### Dendritic Cells (DC)

Dendritic cells (DC) are a unique class of leukocytes whose primary function is to capture, process, and present antigens to T cells (Steinman, Annu. Rev. Immunol. 1991; 9:271-96). They can be found in non-lymphoid tissue, but migrate to the T-dependent areas of lymphoid organs. In non-lymphoid organs, DC include Langerhans cells, blood and mucosal DC. In lymphoid organs, they include lymphoid DC and interdigitating DC. Interaction between DC and specific T cells in the peripheral immune system leads to the induction of immune responses, whereas in the thymus presentation by dendritic cells leads to negative selection (Tanaka et al., Eur. J. Immunol. 1993; 23:2614-2621; Matzinger et al., Nature 1989; 338:74-76).

Dendritic cells (DC) are the most efficient antigen presenting cells (APC) for both CD4+ and CD8+ T cells. The majority of DC are bone marrow-derived cells that are predominantly found in the T-cell rich areas of lymphoid tissue. These cells express high levels of MHC class I and II adhesion and co-stimulatory molecules and mannose receptor-like proteins (Steinman,supra; Metlay et al., J. Exp. Med. 1990; 171:1753-1771; Kraal et al., J. Exp. Med. 1986; 163:981-997; Jiang et al., Nature 1995; 375:151-155). Mature DC can be derived from bone marrow and cord blood as well as PBMC using a variety of cytokines (Inaba et al., J. Exp. Med. 1992; 173:549-559; Inaba et al., J. Exp. Med.1992; 176:1693-1702; Inaba et. al., Proc. Natl. Acad. Sci. USA 1993; 90:3038-3042). Although there are no evident phenotypic differences, mature DC have been shown to present processed antigens to T cells more efficiently than the immature DC. In contrast, the immature DC are efficient in processing endogenous as well as absorbing and processing exogenous proteins and present antigen peptides, but lack expression of the co-stimulatory molecules and cytokines leading to T cell activation.

DC are implicated for the use of anti-tumor and infectious disease vaccines as well as transplantation tolerance. DC are important inducers of both T cell immunity and T cell tolerance (Banchereau et al., Nature 1998; 392:245-252, Viney et al., J. Immunol. 1999; 160:5815-5825). It has been proposed that immature DC which present antigen peptides in the absence of co-stimulation induce tolerance rather than immunity, and it is noteworthy that mucosal tussues are enriched with phenotypically immature DC (Steinman, Fundamental Immunology, Ed. W. E. Paul, 1999: pp. 547-573, Lippincott-Raven Publishers, Philadelphia.) However, the possibility has been raised that immunity and tolerance are instead induced by different subsets of DC: immunity is induced by myeloid DC responsive to granulocyte-macrophage-colony stimulating factor (GM-CSF), and tolerance by lymphoid DC which are not influenced by GM-CSF (Steinman, supra). The enhanced presentation of CTB-conjugated antigens by DC in vitro is therefore in line with both the enhanced antibody responses and the induction of peripheral T cell tolerance in vivo, which have been observed when different CTB-coupled antigens are administered mucosally, and with the consistent immune stimulation by CTB-coupled antigens or DC ex vivo as described hereinbelow. Methods of obtaining and purifying human dendritic cells are well known. See, for example, the methods described hereinbelow and the U.S. Pat. No. 6,004,807 to Banchereau et al. and U.S. Pat. No. 6,194,204 to Crawford et al.

### Macrophages (MØ)

Macrophages (M\acute{Ø}) are generally a population of ubiquitously distributed mononuclear phagocytes responsible for numerous homeostatic, immunological, and inflammatory processes, including a role as APCs. Their wide tissue distribution makes these cells well suited to provide an immediate defense against foreign elements prior to leukocyte immigration. Since macrophages participate in both specific immunity via antigen presentation through the MCH class II restricted pathway, resulting in IL-1 production and nonspecific immunity against bacterial, viral, fungal, and neoplastic pathogens, it is not surprising that macrophages display a range of functional and morphological phenotypes.

The basic mechanism through which MØ function as APCs is presentation of fragments of internalized antigen at the cell surface, nestled in the groove of class II histocompatibility molecules, followed by recognition by a CD4+ T cell that recognizes the displayed antigen, which is then stimulated to release lymphokines.

### B Lymphocytes

B lymphocytes process antigen by the MHC class II pathway. However, antigen processing by B cells differs from that of phagocytic cells like macrophages in crucial ways. First, B cells engulf antigen by receptor-mediated endocytosis. The B cell receptors for antigen are antibodies anchored in the plasma membrane. The affinity of the B cell receptors for an epitope on an antigen may be so high that the B cell can bind and internalize the antigen when it is present in body fluids in concentrations thousands of times smaller than a macrophage would need. The remaining steps of antigen processing occur by the same class II pathway described above for macrophages. These, in turn, stimulate the B cell to enter the cell cycle. Like other APC, B cells also have GM₁ molecules displayed on the cell surface. This allows GM₁-binding molecules such as CT or CTB and their coupled or fused antigens to enter and use B cells as APC through a separate pathway than that by the normal antibody receptors. Therefore, when limited to CT or CTB, all B cells, both naive and memory B cells, can be used as APC.

To which extent B cells contribute to T cell priming leading to antibody responses and/or to peripheral tolerance is not clear. B cells are not required for immune priming in vivo (Epstein et al., J. Exp. Med. 1995; 182:915-922) and turn off rather than activate naive T cells (Fuchs et al., Science 1992; 258:1156-1159). At the same time, some mucosal adjuvants can promote strong antibody responses by simultaneously activating and targeting antigen to B cells (Ågren et al J. Immunol. 1997; 158:3936-3946).

Antigens

The immunogen protein or peptide or part thereof used with CT or CTB is preferentially selected so as to contain either or both of a T-cell epitope and B-cell epitope from the target antigen one wants the direct immune response against. Peptides containing such epitopes have in many cases been described in the literature. Therefore, the information can be obtained from publications to guide the construction of fusion proteins or the purchase of synthetic peptides with the same sequence. In the absence of specific sequence information, T-cell and B-cell epitopes can be identified by standard T-cell proliferation and/or ELISA screening methods using synthetic peptides from the target protein antigen. The size of the B-ell epitope should be sufficient to allow for some degree of folding, and the T-cell epitope should be large enough to encompass the full epitope, typically about 10 amino acid residues in length. Practically, the peptides linked or fused to the toxin carrier exceed 10 and are usually about 20 amino acids, but may be as long as 50 amino acids or more. Optimization can be performed on a case-by-case basis.

For purposes of the present invention, peptides are compounds of two or more amino acids and include proteins. Peptides are preferably of low molecular weight, of about 1,000 kD to about 10,000 kD, more preferably about 1,000 kD to about 5,000 kD. The peptides of the invention may be from about 8 to about 50 amino acids, preferably from about 10 to about 20 amino acids. The peptides of the present invention include but are not limited to peptides which bind to MHC molecules, cell surface-associated proteins, peptides associated with a heat shock proteins/chaperonins, proteins encoded by cancer oncogenes, or mutated anti-oncogenes. In one preferred embodiment of the invention, peptides are bound to the MHC molecules. For purposes of the present invention “a peptide equivalent” is a peptide having the same amino acid sequence as the peptide isolated from an MHC molecule, although prepared either by degradation of a protein comprising the peptide, synthesized in vitro or using recombinant DNA technology.

### Antigens, with Special Reference to Tumor Antigens

For purposes of the present invention, the antigens used in conjunction with CT or CTB or related proteins are preferentially proteins or peptides, but may also include carbohydrate or lipid or complex antigens (e.g., glycolipids, glycoproteins, lipoproteins, etc.), as well as nucleic acids (e.g., DNA or RNA) encoding a protein or peptide antigen. In a preferred embodiment, the antigen is selected from a group of protein or peptide antigens representing relevant targets for vaccination purposes, including but not limited to antigens of infectious agents (e.g., bacterial, viruses, fungi, protozoa, helminthes, prions), tumor cells, and abanormal proteins such a β-amyloid, implicated in Alzheimer's disease and other neurodegenerative disorders. In a particularly preferred embodiment, the antigens are proteins or peptides specific for or over-expressed by tumor cells as compared with normal cells.

Preferably, the protein or peptides represent or are derived from tumor specific antigens. Proteins or peptides may be isolated from the cell surface, cell interior, or any combination of the two locations. The extract may be particular to type of cancer cell (versus a normal cell). There is substantial evidence that the same T cell-defined tumor antigens are expressed by different human tumors, suggesting that transformation-associated events may give rise to recurrent expression of the same tumor antigen in tumors of related tissue and/or cellular origin (Shamamian et al., Cancer Immunol. Immunother., 1994;39:73-83; Cox et al., Science, 1994;264:716). Examples of such tumors include solid and disseminated tumors, but are not limited to lung, colorectal, pancreatic, prostate, ovarian, breast, multiple myeloma, leukemias and melanoma. Examples of tumor-associated antigens include but are not limited to HER2/neu or c-erbB-2, HER3 and HER4 (breast), MAGE-1 and MAGE-3 (bladder, head and neck, non-small cell carcinoma); MART 1/Melan A, gp-100, and tyrosinase (melanoma); carcino-embryonic antigen (CEA-colon, brat, gastrointestinal tract); MUC-1 complex antigens including 19-9 and CA242 (breast, pancreas, colon and prostate); prostate-specific membrane antigen (PSMA); HPV E6 and E7 proteins (cervical cancer); tumor-associated heat-shock protein antigens, and various other glycolipid antigens With sugar specificity including fucosyl-GM1, GD3, and sialyl T (Hakomori, Cancer Res. 1985; 45:2405-14; Nilsson et al., Glycoconjugate J. 1984; 1: 43-49; Nilsson et al., Cancer Res. 1986; 46: 140301407; Fredman et al., Biochim. Biophys. Acta 1986; 875: 316-323).

The tumor antigens described supra can be obtained synthetically or from whole irradiated tumor cells, tumor cell extracts, or membrane preparations as described (Herlyn and Koprowski, Ann. Rev. Immunol. 1988; 6: 283-308; Schreiber et al., Ann Rev. Immunol. 1988; 6: 359-80; Screiber, Fundamental Immunol. Ed. W. E. Paul 1999, pp. 1237-1270, Lippincott-Raven, Philidelphia). An advantage to using tumor cells, extracts or membrane preparation is that the methods of the present invention can be achieved in the absence of known tumor associated antigens or peptides and sequence information thereof.

The conjugates of the invention, including peptides originally isolated from MHC molecules located on tumor cell plasma membranes, have the property of stimulating T cells. For purposes of the present invention, stimulation refers to proliferation of T cells as well as production of cytokines by T cells in response to the cell extract. Proliferation of T cells may be observed by uptake by T cells of modified nucleic acids, such as but not limited to ³H thymidine, ¹²⁵IUDR (iododeoxyuridine); and dyes such as 3-(4,5-dimethylthiazol-2-yl)-2,5-diphenyltetrazolium bromide (MTT) which stains live cells. In addition, production of cytokines such as but not limited to gamma-interferon (IFN-γ), tumor necrosis factor (TNF), and interleukin-2 (IL-2) may be used to monitor for stimulation, for example, using ELISA (available from R & D, Minneapolis, Minn.). Production of cytokines is preferably in an amount greater than 15 pg/ml, more preferably about 20 to about 30 picograms/ml, even more preferably about 50 pg/ml. Alternatively, cytotoxicity assays can be used to evaluate T cell stimulation.

The present invention is further described in working examples, which are indented to further describe the invention without limiting its scope.

### EXAMPLES

The present invention is also described by means of examples, including the particular Examples presented here below. The use of such examples anywhere in the specification is illustrative only and in no way limits the scope and meaning of the invention or of any exemplified term. Likewise, the invention is not limited to any particular preferred embodiments described herein. Indeed, many modifications and variations of the invention will be apparent to those skilled in the art upon reading this specification and can be made without departing from its spirit and scope. The invention is therefore to be limited only by the terms of the appended claims along with the full scope of equivalents to which the claims are entitled.

### Methods

The descriptions and references provided readily permit those of ordinary skill in the art to reproduce the experiments as well as to undertake variations and modifications both with regard to the nature and choice of antigens used, the methods of conjugation or genetic fusion, and the methods used for preparation of cells and immunological assays.

Chemical Conjugation of Peptide or Protein Antigens to CTB or CT

Coupling of influenza virus haemagglutinin peptide to CTB. A synthetic peptide corresponding to amino acid residues 108 119 of influenza virus haemagglutinin H1 subtype (HA peptide) was purchased from Neosystem (Strasbourg, France). Recombinant CTB (rCTB) was produced and purified from V. cholerae strain 358 as described (Lebens et al., Bio Technology 1993; 11:1574-1578). CT was purchased from LIST Biological Laboratories (Campbell, Calif.). Whole ovalbumin (OVA) was purchased from Sigma (St. Louis, Mo., USA).

The HA peptide and OVA protein were chemically coupled to rCTB or CT using N-succinimidyl (3-[2-pyridyl]-dithio) propionate (SPDP Pharmacia) as a bifunctional coupling reagent as described (Rask et al., Clin. Exp. Allergy 2000; 30:1024-1032). Conjugated material was quantified and purified by FPLC gel filtration (Superdex 200 16/60 column, Pharmacia Biotech, Uppsala, Sweden). The conjugates were analyzed in a GM1-ELISA using biotinylated anti-CTB monoclonal antibodies (Svennerholm et al., Curr. Microbiol. 1978; 1:19-23), and were shown to have retained GM1-binding activity.

Coupling of ovalbumin or tetanus toxoid to CT or CTB. Tetanus toxoid (TT) prepared by formalin inactivation of tetanus toxin for use in human vaccine against tetanus was obtained as a gift from SBL Vaccin, Stockholm, Sweden. CT and rCTB were produced and purified as described supra.

OVA or TT protein were chemically coupled to CT or rCTB, quantified and purified as described above. The purified conjugates contained equimolar concentrations of OVA to CT and CTB, respectively. The conjugates were analyzed in a GM1-ELISA using biotinylated anti-CTB and anti-CTA monoclonal antibodies as well as polyclonal antisera and with polyclonal mouse or rabbit antisera to OVA and TT and were shown to have retained reactivity with GM1 as well as with anti-CTB, anti-OVA or anti-TT as appropriate and for the CT-conjugates also with anti-CTA antibody.

Construction and Purification of CTB Fusion Proteins

Methods have been described for making fusion proteins based on CTB or LTB wherein nucleic acids encoding for either or both of T or B epitopes of an antigen of interest are genetically fused to coding sequences for either or both of the N- or C-terminus of CTB, or placed in an intrachain position in the CTB or LTB coding sequence, or to analogous positions in CTA or LTA (Bäckström et al., Gene 1995; 165:163-171, Bäckström et al., Gene 1994; 149:211-217, Schödel et al., Gene 1991; 99:255-259). Methods have also been described for fusing peptides to the carboxy or amino ends of CTA or LTA and for co-expressing these fusion proteins with CTB or LTB (Sanchez et al. FEBS Lett. 1986; 208:194-198, Sanchez et al. FEBS Lett. 1997; 401:95-97).

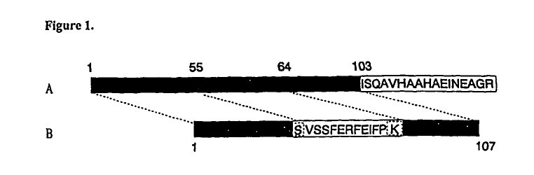

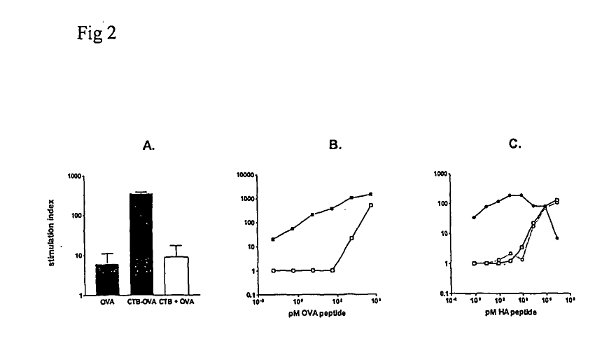

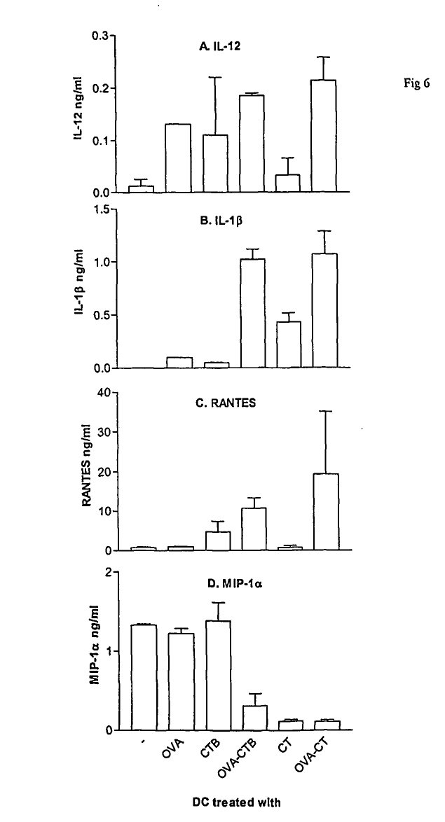

For use in examples described below, two such recombinant CTB fusion molecules were made, one in which peptide 323-339 of OVA was fused to the C terminus of CTB (CTB::OVAp, FIG. 1A), and one in which a peptide corresponding to the influenza virus haemagglutinin residues 108-119 (HA) replaced residues 56-63 in the CTB structure (CTB56 63Hap.

CTB::OVAp: Synthetic oligonucleotides encoding the 17 amino acid OVA peptide were synthesised (Innovagen AB, Lund, Sweden). The oligonucleotides were annealed and then ligated onto the 3′ end of a CTB gene. DNA sequencing of the final plasmid, pML CTB::OVA, confirmed the final sequence of the gene fusion.

CTB56 63HAp: Synthetic oligonucleotides encoding the 12 amino acids HA peptide were synthesised (Innovagen AB). The oligonucleotides were inserted into plasmid pCB56 63gp12 (Bäckström et al., Gene 1995; 165:163-171) between positions 55 and 64 of mature CTB. DNA sequencing was used to confirm the sequence of the insert in pCB56 64HA.

Recombinant proteins carrying peptides inserted into CTB are more stable than those carrying peptides linked to the N or C termini (Bäckström et al., Gene 1994 149:211-217). Thus, whereas CTB56 63HAp could be expressed in Vibrio cholerae, CTB::OVAp was expressed in E. coli to avoid cleavage by extracellular V. cholerae proteases known to readily destroy C terminal peptide extensions on CTB (Schödel et al., Gene 1991; 99:255-259).

pCB56 63HAp was transferred into V. cholerae strain JS1569 by electroporation (Lebens et al., Bio Technology 1993; 11:1574-1578). The protein was precipitated from the growth medium using hexametaphosphate (Lebens et al. Bio Technology 1993; 11:1574-1578), and was then re-dissolved in a minimal volume of 0.2 M Tris HCl pH 8.0 and dialysed against PBS pH 7.2.

pML CTB::OVA was transferred into E. coli BL21 (Grodberg et al. J. Bacteriol. 1998; 170:1245-1253). The CTB gene used lacked the signal peptide directing transport of the synthesised protein into the periplasmic space. This resulted in the cytoplasmic accumulation of the product (CTB::OVAp) as monomers, which formed insoluble inclusion bodies. These were dissolved in 6.5M urea and reassembled by dialysis (L'Hoir et al, Gene 1990; 89:47-52).

The CTB::OVAp and CTB56 64HAp fusion proteins were further purified by ion exchange (Resource Q column, Pharmacia Biotech, Uppsala, Sweden) and FPLC gel filtration (Superdex 200 16/60 column, Pharmacia Biotech of Uppsala, Sweden) using the Biologic Workstation FPLC system (Biorad of CA, USA).

The CTB fusion proteins were shown to have retained GM1 binding activity by means of GM1 ELISA using biotinylated anti CTB monoclonal antibodies.

Preparation of Mouse APC and Other Cells and Mouse Immunological Assays

Detailed descriptions for all methods used which are not described otherwise in the protocols below can be found in Current Protocols in Immunology, ed. J. E. Coligan et al. John Wiley & Sons. Inc. 1997, volume 1, to which the reader is referred.

Generation of APC. For the generation of bone marrow derived DC and macrophages (MO), male BALB/c mice (B&K Universal AB, Stockholm, Sweden) were killed and bone marrow was flushed from the femur and tibia and depleted of erythrocytes with ammonium chloride. For preparation of “total APC” total spleen cells were used after removal of T cells by standard neuraminidase-treated sheep red blood cell resetting as described (Current Protocols in Immunology, ed. J. E. Coligan et al. John Wiley& Sons. Inc. 1997, Volume 1).

DC were generated from bone marrow precursors as described (Inaba, K., M. Inaba et al. J. Exp. Med. 1992; 176:1693-1702). Briefly, after the removal of erythrocytes, T-cells, B-cells and MHC II positive cells were removed by incubating with 1 μg/ml each of rat anti-mouse CD4, CD8, B220 and I-Ad antibodies (Pharmingen) and anti-rat IgG-coated and anti-mouse IgG-coated beads (Dynal). Remaining cells were plated in 24-well plates (10⁶ cells/well) in Iscoves medium supplemented with 10% FCS and 1125 U/ml recombinant murine GM-CSF (Pharmingen) (referred to as DC medium). Half of the DC medium was replaced every second day. On day 6, non-adherent cells were collected and further purified by metrizamide density centrifugation (18% metrizamide in PBS; Sigma) at 800×g. More than 90% of the DC obtained were CD11c positive. In some experiments, DC were further purified using anti-mouse CD11c microbeads (Miltenyi Biotec, Bergisch Gladbach, Germany) according to the manufacturer's instructions, yielding >99% pure CD11c-positive DC.

To generate macrophages (Warren et al. J. Immunol. 1985; 134:982-989), plastic adherent cells were removed from bone marrow precursor cells by incubation in a 20 ml flask over night at 37° C. and the remaining cells were cultured at 5×10⁵ cells/ml in 75 cm² flasks (20 ml/flask) in complete medium containing 20 ng/ml of colony stimulating factor 1 (Sigma). Adherent macrophages were retrieved on day 7 by a 5 min incubation at 37° C. with 5 ml PBS containing 2.5 U dispase I (Boehringer Mannheim).

B cells were purified from spleen cell suspensions after initial depletion of plastic adherent cells by using B220 specific magnetic beads (Miltenyi Biotec, Germany) which generated >90% pure B220 positive cells as analyzed by FACS (see below).

Purification of HA specific TCR transgenic and non-transgenic T cells. T cells were purified from peripheral lymph nodes or spleens of naive BALB/c mice expressing a transgenic alpha/beta chain T cell receptor (TCR) specific for peptide 111-119 of influenza virus haemagglutinin (HA) in the context of I Ed (19-kind gift from Dr H. von Boehmer, Harvard, USA) and BALB/c mice expressing a transgenic alpha/beta T cell receptor (TCR) specific for peptide 323-339 of OVA (32-kind gift from Dr Nils Lycke, University of Göteborg) using T cell purification columns (R&D) followed by panning on Petri dishes coated with anti IAb,d monoclonal antibodies (5 mg/ml, Pharmingen). More than >98% of the resulting T cell population were CD3+ of which approximately 10% expressed the transgenic TCR in HA TCR animals and 50-70% expressed the transgenic TCR in OVA-TCR animals. T cells from peripheral lymph nodes or spleens of naive or immunized non-transgenic BALB/c mice (B&K Universal AB, Stockholm, Sweden) were purified and characterized by similar methods as those used for the TCR-transgenic T cells using T cell purification columns (R&D, Minneapolis, Minn.) followed by panning on Petri dishes coated with anti IAb,d monoclonal antibodies (5 mg/ml, Pharmingen). More than >98% of the resulting T cell population were CD3+ as tested by FACS.

FACS analysis. APC were analysed either a) immediately following isolation/in vitro generation, or b) 24 hours after 10⁶ antigen pulsed APC had been incubated either alone or together with 10⁶ antigen specific TCR transgenic T cells in flat bottomed 24 well plates (Nunc). Cells were analyzed by FACS using the following antibodies from Pharningen: FITC anti-I Ab clone 25 9 17, FITC anti mouse CD40 clone HM40 3, PE anti mouse CD80 clone 16 10A1, PE anti mouse CD86 clone GL1, PE anti mouse B220 clone RA3 6B2, and PerCP anti mouse CD3e clone 145 2C11. Antigen-pulsed DC also were analyzed using murine anti-OVA serum (1/10 dilution) or with a monoclonal antibody to CTB (LT39; supernatant at a 1/10 dilution (Rappuoli et al., Immunol. Today 1999; 20:493-500) for 30 min at 4° C. followed by FITC-labelled goat anti-mouse IgG (Serotec). TCR transgenic T cells were analyzed using PE anti-mouse CD3e clone 145-2C11 from Pharmingen and a FITC-labeled rat clonotypic monoclonal antibody 6.5 recognizing the HA-specific transgenic TCR (Weber et al., Nature 1992; 356:793-796) or FITC-labeled KJ1-26 mAb recognizing OVA-specific transgenic TCR (Marrack et al., J. Exp. Med. 1983; 158:1635-1646).

Proliferation assays. APC were irradiated at 900 rad and then incubated with graded amounts of antigen for 90 min at 37° C., extensively washed, and plated in triplicates at 10⁴ or 10⁵ cells/well in flat bottomed 96 well plates (Nunc) together with 10⁵ HA specific or OVA specific transgenic T cells in complete medium. Plates were incubated for 2-3 days at 37° C. Culture supernatants were collected at 48 hours and frozen at 70° C. until assayed for cytokine content. 1 mCi (⁶⁻³H) thymidine (Amersham, UK) was added to each well 8 hours before harvesting and incorporated radioactivity was measured. Results are expressed either as antigen specific ³H thymidine incorporation (cpm), or as stimulation indexes (SI) defined as the ratio between ³H thymidine incorporated into T cells incubated with antigen treated APCs and ³H thymidine incorporated into T cells incubated with mock treated APCs.

Cytokine measurements. DC were incubated at 10⁵ cells/well in flat-bottomed 96 well plates (Nunc) in the presence or absence of specific antigen in conjunction with CT (0.05 μM) or CTB (0.5 μM) in 200 μl complete medium. Culture supernatants were collected at 24 hours or later and frozen at −70° C. until assayed for cytokine content. Culture supernatants were analysed for IL-1β, RANTES and MIP-1α content using specific Duoset ELISAs from R&D according to instructions. IL-12 and IL-18 were similarly measured using OptEIA mouse IL-12 (p40) and IL-18 sets from Pharmingen (San Diego, USA).Culture supernatants were analysed for IL-1β and IFN gamma content using Duoset ELISAs for mouse IL-1β and IFN-γ from R&D according to instructions. IL-12 and IL-18 were similarly measured using OptEIA mouse IL-12 (p40), IL-12 (p70) and IL-18 sets available from Pharmingen (San Diego, USA). IL-4 and IL-10 were measured either by ELISA or through a more sensitive modified cell ELISA method using specific antibody pairs from Pharmingen (Beech et al., J. Immunol. Methods 1997; 205:163-168). The sensitivity of both assays was 30 pg/ml.

Ex vivo and in vivo vaccinations and sample collection. DC (or other types of APC) were pulsed for 2 hours at 37° C. with OVA, OVA conjugated to CTB (OVA-CTB) or OVA conjugated to CT (OVA-CT) at equimolar concentrations of OVA (0.5 μM or 0.05 μM), in the presence or absence of CT (0.05 μM or CTB (0.5 μM), and then extensively washed by 3 rounds of centrifugation prior to injection.

Balb/c mice were given two intravenous injections (or for comparisons two subcutaneous or intraperitoneal injections), two weeks apart, of 2×10⁶ DC (or other types of APC) in 100 μl PBS. Two weeks after the second DC vaccination, mice were boosted with 3 μg OVA in FCA subcutaneously. Serum was taken prior to vaccination, at the time of booster (d14 post DC2), and on day 9 post-OVA-FCA, and frozen at −20° C. until analyzed for OVA-specific antibody production. Mice were sacrificed day 9-14 post the OVA booster injection. The spleen was collected and single cell suspensions were analyzed for OVA-specific IFN-γ production in vitro. These studies were approved by the Ethical Committee for Animal Experimentation (Goteborg, Sweden).

Antibody responses in serum. High-binding microtiter plates (Greiner) were coated with OVA (20 μg/ml in PBS) overnight. Serial dilutions of sera was incubated for 90 min in PBS containing 0.1% BSA. Thereafter peroxidase-conjugated anti-mouse IgG (1/3000), IgG1 (1/1000) or IgG2a (1/1000) (all from Southern Biotechnology) were added for 90 min at RT. The reaction was develop with 100 μl of 1 mg/ml O-phenylene diamine dihydrochloride (Sigma) in 0.1M citrate buffer pH 4.5 containing 0.04% H₂O₂, and read at 450 nm. The specific antibody titer was estimated as the interpolated sample dilution giving an absorbance of 0.4 above the background level. No anti-OVA antibodies were detected in pre-immunization sera. Data are expressed as the mean reciprocal titer+SD for 3 or more mice.

Cytokine measurements using T cells from DC-immunized animals. T-cell analyses from DC-immunized animals were hampered as the DC induced FCS-specific T-cell responses. To circumvent this problem we used another source of serum (horse serum) in our in vitro analysis which, in our hands, only allowed short time assays (i.e., cytokine determinations but not proliferation assays).

Measurement of IFN-γ production in spleen cells was performed using a modified version of a cell ELISA method (Beech et al. J. Immunol. Methods 1997; 205:163-168) using an IFN-γ ELISA kit (R&D, Minneapolis, Minn.) according to instructions. Briefly, mononuclear spleen cell suspensions were seeded in duplicate at different cell densities in Iscoves medium containing 5% horse serum in the presence or absence of OVA (500 μg/ml) and incubated at 37° C. for 24 h in anti-IFN-gamma coated flat-bottom 96 well plates. The cells were removed by extensive washing and biotinylated anti-mouse IFN-gamma was added overnight at 4° C. followed by a 45 min incubation with 2 μg/ml peroxidase-labelled avidin (Sigma, St. Louis, Mo., USA) at room temperature. Color development was achieved with peroxidase substrate containing 3,3′,5,5′-tetramethylbenzidine (0.1 mg/ml, Sigma) and 0.06% H₂O₂ in 0.05 M phosphate-citrate buffer at pH 5.0. The reaction was stopped using 25 μl 1M H₂SO₄ and the absorbance was read at 450 nm. The concentration of IFN-γ was determined by extrapolation from a standard curve using recombinant cytokine. Results are expressed as the concentration of IFN-γ secreted per one million spleen cells. In one experiment we depleted the spleen cell suspensions of either CD4+ cells or CD8+ cells prior to analysis using rat anti-mouse CD4 and rat anti-mouse CD8 antibodies (Pharmingen), respectively, and anti-rat IgG-coated beads (Dynal).

Preparation of Human APC and Other Cells and Human Immunological Assays Used

Different types of human APC, including DC, monocyteslmacrophages, B cells, and “total APC,” were purified from peripheral blood mononuclear cells (PBMC) prepared from heparinized human venous blood as described below. Alternative isolation sources include, for example, peripheral blood “buffy coat”, bone marrow aspirates, surgically removed lymph nodes, pleura fluid, and surgical tumor and epithelial tissue specimens.

Dendritic cells. For generation of DC, PBMC were prepared from heparinized venous blood by Ficoll-Hypaque (Pharmacia, Uppsala, Sweden) density gradient centrifugation as described (Current Protocols in Immunology, ed. J. E. Coligan et al. John Wiley & Sons. Inc. 1997, volume 2). Cells, 1×10⁶ cells per well, were then incubated for 2 hours at 37° C. in 6-well plastic tissue culture plates in Iscove's complete medium supplemented with 5% autologous (or human AB) serum to allow for attachment of adherent cells to the plastic. Non-adherent material was discarded and the adherent cells were then incubated in Iscove's complete medium supplemented with 5% autologous (or human AB) serum, 1% gentamicin, 1% L-glutamine plus 800 U/ml GMCSF and 500 U/ml IL-4 (Pharningen)—referred to as DC medium—at 37° C. for 6-7 days with exchange of half of the medium every second day. In alternative procedures, CD14+ cells were first isolated from the PBMC using anti-CD14 coated magnetic beads and separation columns as described by the manufacturer (CD14 microbeads and LS separation columns from Miltenyi Biotec GmbH). To achieve further mature DC, the DC medium used for the last 2 days of incubation was in some tests further supplemented with TNF-α 100 U/ml and/or so-called monocyte-conditioning medium obtained by culturing PBMC in an anti-immunoglobulin coated plate for 2 days and then using the supernatant medium.

Monocytes/MØ. Monocyte/macrophage populations were isolated from PBMC using the plastic adherence method as described (Current Protocols in Immunology, ed. J. E. Coligan et al. John Wiley & Sons. Inc. 1997, volume 2, 7.6.1 basic protocol) with the modification that the PBMC were allowed to adhere in Iscove's medium supplemented with 5% autologous (or human AB) serum. For further purification, the detached adherent cells were subjected to size sedimentation by centrifugation through a Percoll gradient as described (Current Protocols in Immunology, ed. J. E. Coligan et al. John Wiley & Sons. Inc. 1997, volume 2, 7.6.2 basic protocol) but using autologous serum (or human AB serum) in stead of fetal calf serum.

B lymphocytes. B cells were isolated from PBMC using immunomagnetic positive selection using anti-CD19 coated MACS-beads (Dynal) as described (Current Protocols in Immunology, ed. J. E. Coligan et al. John Wiley & Sons. Inc. 1997, volume 2; 7.5.3 alternate protocol).

“Total APC” were prepared from PBMC by depletion of T cells using rosetting with neuraminidase-treated sheep red blood cells (SRBC) as described (Current Protocols in Immunology, ed. J. E. Coligan et al. John Wiley & Sons. Inc. 1997, volume 2, 7.2.1 basic protocol).

For storage prior to use, isolated APC can be frozen for up to several weeks, or placed in methylcellulose gel for several days.

Purification of T cells. Peripheral blood mononuclear cells (PBMC) were isolated from venous blood by Ficoll-Hypaque (Pharmacia, Uppsala, Sweden) density centrifugation. PBMC in Iscove's medium supplemented with 10% FCS and antibiotics (culture medium) were incubated in Petri dishes (2×10⁷ cells/Petri dish) for 90 min at 37° C. T cells were purified using resetting with neuraminidase-treated sheep erythrocytes (SRBC) as described (Coligan et al. Current Protocols in Immunology, vol. 2, sect. 7.2 John Wiley& Sons. Inc. 1997); cells were checked with FACS to contain >95% CD2+ T cells.

Ex vivo treatments of APC. DC or other APC were incubated (“pulsed”) for 2 hours at 37° C. with antigen (TT) alone, or antigen conjugated to CTB (TT-CTB) or to CT (TT-CT), or antigen mixed with CT (0.1 or 0.01 μ/gml) or CTB (0.1 μg/ml) [TT+CT or TT+CTB] using equal concentrations of TT (0.5 microgram/ml) and then extensively washed by 3 rounds of centrifugation. Membrane filters can also be used to wash the APC. Before use in T cell proliferation tests, see below, the APC were irradiated with 2000 rad.

In vitro proliferation assay. 96-well flat-bottomed microwell plates (Nunc) were used. 7.5×10⁴ T-cells were added per well together with one or the other type of 2000-rad treated APC (DC, B cells, monocytes or “total APC”) exposed to antigen alone or in conjunction with CT or CTB as specified in the examples. Plates were incubated for 5 days at 37° C. with 7.5% CO². 1 μCi (⁶⁻³H)thymidine (Amersham, UK) was added to each well 16 hours before harvesting and incorporated radioactivity was measured. Results are expressed as mean cpm values of experiments performed in triplicates.

### Example 1

### Coupling of Antigen to CTB Enhances Antigen Presentation In Vitro by Unfractionated “Total APC”

To examine the effect of CTB as carrier protein on antigen presentation by different APC, the antigens were coupled to CTB, either chemically or genetically, and incubated with APC. Using whole spleen cell suspensions as APC we found that conjugation of proteins or peptides to CTB greatly reduced the antigen concentration required for effective presentation to cognate TCR transgenic T cells (FIG. 2). When APC were pre-incubated with a fixed concentration of OVA or CTB-OVA, corresponding to 10⁻⁸M OVA, prior to presentation to OVA-specific TCR-transgenic T cells, the CTB-OVA conjugate gave rise to a 80-fold higher proliferative response than that obtained with free OVA (FIG. 2A). Similarly, the CTB fusion protein carrying OVA peptide (CTB::OVAp) was effective at a >10⁴-fold lower concentration as compared to the concentration of free OVA peptide required (FIG. 2B). Similar results were obtained when a HA peptide genetically fused to CTB was compared to a free HA peptide, using HA-specific TCR-transgenic T cells as the readout system (FIG. 2C). The proliferative responses obtained were antigen specific as T cells from wt BALB/c animals did not proliferate in response to either HA or OVA peptide or to the corresponding CTB derivatives (not shown).

There was a requirement for a physical interaction between the antigen and the CTB molecule for the beneficial effect on antigen presentation to occur, since co-administration of free CTB and free antigen did not result in any enhancement of the T cell proliferative responses as compared to those obtained with antigen alone (FIG. 2A, C).

### Example 2

### Conjugation of Antigen to CTB Enhances Antigen Presentation by Different Types of Purified APC: DC, B-Cells and MØ

In this example, three different sources of APC were used that were either generated from bone marrow precursors grown in the presence of specific cytokines to differentiate into either DC or MØ, or B cells purified from the spleen of naive BALB/c animals. The different types of APC were incubated for 90 min with the HA or OVA peptides, OVA, or their CTB fusion protein derivatives, and then extensively washed and added at a 1:10 cell ratio to cultures of purified T cells from HA- or OVA-specific TCR transgenic animals. Data were generated from one of two experiments using 10⁵ purified T cells with 10⁴ APC and expressed as the stimulation index obtained with different concentrations (10⁰, 10² and 10⁴ nM). Under these conditions, DC and B cells, which constitutively express MHC II, were efficient APC for free peptide (OVA and HA), chemically conjugated CTB-peptide (OVA and HA), genetically coupled CTB-HA peptide, or whole protein (OVA), antigens requiring approximately 10 and 100 nM free antigen respectively to induce a detectable in vitro T cell proliferative response. Conversely, MØ treated with free peptide or protein antigens were not able to induce any significant proliferation by the HA- or OVA-specific TCR-transgenic T cells using free or linked peptide or protein. These results are in accordance with FACS data showing significant MHC II expression on the DC and B cells, but not on MØ (not shown).

The proliferative responses to CTB linked antigens were strongest using DC or B cells as APC, which achieved a stimulation index of between 100-1000 for HA at a concentration of 100 nM, and between 100 and 10000 for OVA. However, conjugation of antigen to CTB also induced strong T cell proliferation using MØ as APC, achieving a stimulation of about 100 for CTB-HA and 400 for CTB-OVA. Both genetic and chemical CTB-peptide conjugates could be utilized for this purpose, and CTB-conjugation was shown to be an efficient means of enhancing the subsequent proliferative response to both peptide (OVA peptide) and protein (OVA) antigens.

### Example 3

### Conjugation of Peptide to CTB Enhances the Proliferative Response of Peptide-Specific T Cells and Induces Enhanced IFN-γ and IL-12 Production

The enhanced antigen presentation obtained with CTB-conjugated antigen could be blocked if anti-CTB immune serum from mice (used at 1/100) or GM1 (10 mM) were included during the pre-incubation step (not shown) showing that the enhanced antigen presentation was dependent on CTB-mediated binding to GM1 receptors on APC. The enhanced antigen presentation results in the production of pro-inflammatory cytokines and hence, an enhanced immune responses. We found that CTB-conjugated antigens induce enhanced IFN-γ and IL-12 secretion in vitro. The ability of different APCs to induce IFN-γ production when pulsed with free or CTB-coupled antigens was compared. IFN-γ secretion was measured in 48 h supernatants from cultures of OVA-specific or HA-specific TCR-transgenic T cells incubated with APC that had been pre-treated with different concentrations (10-10⁴ nM) of OVA, OVA peptide or HA peptide, or with CTB derivatives thereof.

We also observed that different APCs have different potencies regarding eliciting cytokine production. The effect of HA peptide versus CTB-coupled HA peptide on IFN-γ production when DC, B cells or MØ were used as APC was compared. HA peptide alone induced low levels of IFN-γ when DC or MØ were used as APC, unless given at very high dose (about 2000 and 200 pg/ml IFN-γ at 10⁴ nM, respectively)s, and did not induce IFN-γ when B cells were used as APC. When HA peptide had been chemically or genetically coupled to CTB, the levels of secreted IFN-γ were considerably higher, about 2000 and 5000 ng/ml for DC and MØ, respectively, and occurred at >1000 fold lower concentrations (10¹ nM). Furthermore, CTB-coupled HA peptide also induced measurable IFN-γ responses (about 900 pg/ml) when B cells were utilized as the source of APC.

Further, genetic and chemical CTB-HA constructs were compared. In general, the difference between the genetic and chemical CTB-HA constructs in their ability to induce IFN-γ responses was small. However, when MØ were used as APC, the genetic construct was superior to the chemical conjugate, resulting in about 5500 pg/ml IFN-γ production at 10² nM as compared with about 300 pg/ml at the same concentration. When a lower dose of APC was used (10⁴ APC/well), the amount of IFN-γ produced was generally lower although the pattern of secretion was the same as when using the higher APC dose.

Next, the effect of CTB conjugation of a peptide (OVA peptide) and a protein (OVA) antigen was compared. It was found that similar concentrations of free OVA or OVA peptide were required to induce IFN-γ responses by DC, but that OVA consistently induced higher levels of IFN-γ than did the peptide. Neither OVA nor the OVA peptide could induce any measurable IFN-γ responses when B cells were used as APC. Similarly to the CTB56-63HA fusion protein, CTB::OVA hybrid protein was far superior to free OVA peptide at inducing IFN-γ responses, with respect both to the dose of the antigen required (10⁻² vs. 10² nM for DC and 10² vs. 10¹ for MØ) and to the magnitude of the IFN-γ response obtained (about 4500 vs. about 500 pg/ml for DC and about 4000 vs. about 12000 pg/ml for MØ). When CTB was chemically conjugated to native OVA, the IFN-γ responses did occur at a lower OVA concentration, but the differences were not as prominent as those seen with peptide antigens.

Cultures were also analyzed for the presence of Th2 cytokines, e.g., IL-4 and IL-10, but there were no measurable levels of any of these cytokines irrespective of the source of APC or the nature of the antigen.

IL-12 is an important factor in inducing IFN-γ responses (O'Garra, A., Immunity. 1998; 8:275-283). Therefore the levels of IL-12 in the culture supernatants analyzed above for IFN-γ were measured. Data were generated using one of two experiments using 10⁵ purified T cells with 10⁵ APC. Non-conjugated antigen induced substantial levels of IL-12p40 when DC were used as APC. Thus, HA peptide, OVA peptide and OVA at 10 nM gave rise to stong IL-12p40 responses (about 1000 pg/ml), which could be induced at even lower antigen doses when the antigen was coupled to CTB. IL-12p70 could also be detected in these cultures. The pattern of IL-12p70 secretion was similar to that of IL-12p40, but the levels detected were considerably lower. Antigen-pulsed MØ secreted low but measurable levels of IL-12p40 (about 100 pg/ml) and these levels increased when CTB-conjugated antigens were used (about 400 pg/ml for HA and about 3500 pg/ml for OVA). Irrespective of the antigen formulation or the detected levels of IFN-γ, there were only negligible levels of IL-12 in cultures containing B cells as APC. Furthermore, both MØ and DC secreted low levels of IL-12 after exposure to CTB-conjugated antigens also in the absence of specific T-cells.

The CTB56-63HA fusion protein and chemical conjugate between CTB and HA peptide induced similar levels of IL-12p40 from DC (about 2000 pg/ml), whereas the genetic constructs were superior at inducing IL-12p40 production in MØ (about 400 vs. about 50 pg/ml).

As with IFN-γ production, lower doses of APC (10⁴ APC/well) gave a similar pattern of IL-12p40 secretion, but 4-10 times lower than that obtained with 10⁵ APC. Furthermore, all IL-12p40 responses were dependent on the presence of T cells, as no IL-12p40 could be detected in pure APC cultures.

### Example 4

### In Vitro Treatment of DC with Antigen Admixed with or Linked to CT or CTB Before Administration to an Animal Enhances Their Capacity to Induce a Humoral Immune Response In Vivo

It has been postulated that CT exerts much of its adjuvant properties through a direct effect on antigen-presenting cells (Braun et al., J. Exp. Med. 1999; 189:541, Gagliardi et al., Eur. J. Immunol. 2000; 30:2394, 59. Cong et al., J. Immunol. 1997; 159:5301). We therefore tested if CT adjuvant would influence the antibody responses following DC vaccination.

To investigate if treatment ex vivo of DC with antigen in conjunction with CTB or CT, either with antigen conjugated to CTB or CT or admixed with these agents, could induce or potentiate a humoral immune response in vivo after re-infusion of the ex vivo-treated cells (“DC vaccination”), mice were injected twice intravenously with syngeneic DC that were pulsed with free peptide or peptide admixed or linked with CT or CTB. Using similar methods as described in the previous examples, DC were generated from bone marrow stem cells of Balb/c mice and then pulsed for 2 h at 37° C. with either OVA alone (0.5 μM, 0.05 μM OVA admixed with either 0.05 μM CT or 0.5 μM OVA admixed with 0.5 μM CTB, OVA chemically coupled to CT or CTB (0.05 μM CT or 0.5 μM CTB), or only PBS. After washing, the differently treated DC were then injected i.v. to groups of three Balb/c mice, 2 million DC per animal. Two weeks later the mice received a second, identical dose of freshly prepared, and identically treated and washed DC. Finally, after another two weeks the mice were given a s.c. booster injection with 3 μg OVA in FCA. Sera were collected from all animals before the first DC dose, two weeks after the second DC injection just before the s.c. boosting, and 9 days after the final s.c. booster injection, and the levels of OVA-specific IgG antibodies determined by ELISA.

The results are presented in Table I below and show that while the group of animals who had received two doses of PBS-treated DC had not developed any significant anti-OVA antibody response even 9 days after a s.c. immunization with OVA, all groups of mice given two doses with OVA-pulsed DC before the s.c. OVA booster injection had developed a significant serum antibody response to OVA by 9 days after boosting. However, while this response was relatively modest, and barely detectable before the s.c. boosting, in the group of mice that received DC pulsed with OVA alone, much stronger responses were obtained in the groups that received DC pulsed with OVA admixed with either CT or CTB and especially pronounced in the groups that received OVA linked to CT or CTB (p<0.01). In the latter groups, substantial anti-OVA antibody titers had developed in response to the treatment with the pulsed DC per se, and this response was much further increased by the subsequent s.c. boosting with OVA.

To ascertain that DC were responsible for the in vivo priming, we purified CD11c positive cells from our bone marrow cultures and pulsed these cells, as well as non-purified bone-marrow-derived DC, with OVA-CIB prior to vaccination. Both populations of cells primed for B-cell (IgG) and T-cell (proliferative) responses. There were no statistically significant differences in anti-OVA serum titers or OVA-specific IFN-γ responses between the 2 groups of recipient mice.

From these results it is clear that both CT and CTB enhance the in vivo immunogenicity of DC pulsed with specific antigen in vitro and that this effect is especially strong when the antigen is coupled to either of these proteins when presented to the DC.

TABLE 1

| DC | Anti-OVA IgG titer, median | | |
|---|---|---|---|
| treatment | Before 1st DC dose | After 2nd DC dose | After s.c. boost |
| Medium | <50 | <50 | 100 |
| OVA alone | <50 | 500 | 11,000 |
| OVA + CTB | <50 | 2400 | 17,000 |
| OVA-CTB | <50 | 35,000** | 130,000* |
| conjugate | | | |
| Same + CT | <50 | 130,000** | 150,000* |
| OVA + CT | <50 | 30,000** | 32,000 |
| OVA-CT | <50 | 130,000** | 140,000* |
| conjugate | | | |

Data are expressed as the median reciprocal titer for 3 or more mice.
**= p < 0.01 and
*= p < 0.05 as compared to mice receiving antigen-pulsed DC in the absence of CT or CTB.

For comparative purposes, we also examined whether also ex vivo treatment of other APC than the in vitro generated DC with antigen in conjunction with CTB could prime for or induce an immune response similar to that induced by DC in the above experiments. This was done by testing whether also ex vivo treatment of “total APC” and by pulsing with antigen in conjunction with CTB or CT could prime for or induce a humoral immune response, similar to that observed for DC described supra. Total APC comprising total spleen cells after depletion of T cells were pulse-treated with either free OVA or CTB-OVA or CT-OVA ex vivo and then used for vaccinating mice in the same way as described above for DC. Results showed that whilst APC treated with OVA alone did not induce any detectable antibody response by itself and primed for only a modest antibody response after boosting with OVA/FCS, APC treated with either CTB-OVA or even stronger with CT-OVA both by themselves induced significant anti-OVA antibody responses (although titers were 1-2 logs lower than obtained with DC) and primed the animals for high-titered antibody responses after boosting with OVA/FCS (resulting in titers only about a 5-fold lower than those obtained with correspondingly treated DC).

It was also tested whether ex vivo treated APC could be effectively administered through other routes than by intravenous injection. DC were therefore treated ex vivo with CTB-OVA as described and injected twice in mice by the standard intravenous route or subcutaneously or intraperitoneally and antibody responses measured before and after boosting with OVA/FCS as before. Results showed that responses to the subcutaneously or intraperitoneally administered DC were fully as strong or stronger as after intravenous administration.

### Example 5

### CT Treatment of DC Promotes Mixed Th1 and Th2 Responses and CTB Treatment Promotes Th2 Responses

The adjuvant effect of CT has been reported to be Th2 biased (Braun et al., J. Exp. Med. 1991; 189:541, Gagliardi et al., Eur J Immunol 2000; 30:2394). However, in contrast to this we found that CT instead promotes Th1 development. Thus, whereas vaccination with OVA-pulsed or OVA-CTB pulsed DC preferentially induced anti-OVA IgG1 responses (indicative of a Th2 type response), vaccination with CT-treated DC also induced strong anti-OVA IgG2a responses (indicative of a Th2 type response) (Table 2).

Furthermore, when spleen cell suspensions from these mice were activated in vitro with OVA and the IFN-γ production was measured, we found that vaccination using CT adjuvant-treated DC enhanced the OVA-specific IFN-γ responses using both OVA-pulsed and OVA-CTB-pulsed DC (Table 2). The OVA-specific IFN-γ production was more than 4 times higher (p<0.01) following vaccination with OVA-CT-pulsed DC as compared to vaccination with OVA+CT-pulsed DC or OVA-CTB-pulsed DC (Table 2).

TABLE 2

| | Median IgG1/ | Median | |
|---|---|---|---|
| | IgG2a | IgG1/IgG2a anti- | |
| | anti-OVA | OVA titers after | IFN-gamma |
| | titers after | OVA/FCA | production |
| DC treatment | 2^{nd} DC dose | booster | ng/million cells |
| Medium | <50/<50 | <50/<50 | <0.05 |
| OVA | <50/<50 | 150/<50 | 0.2 |
| OVA + CT | 10,000/5,000 | 11,000/5,000 | 0.6 |
| OVA-CTB | 10,000/100 | 60,000/1,500 | 1.0 |
| OVA-CTB + CT | 50,000/12,000 | 60,000/40,000 | 1.9 |
| OVA-CT | 12,000/30,000 | 20,000/50,000 | 2.6 |

### Example 6

### CT and CTB Enhance Expression of Co-stimulatory Molecules and Have Differential Effects on the Production of Cytokines and Chemokines by Pulsed DC

The levels of MHC and co-stimulatory molecules on the surface of the APC influence not only the induction of a T cell response but also the magnitude and the cytokine pattern of that response (Rask et al., Clin. Exp. Allergy 2000; 30:1024, Kuchroo et al., Cell 1995; 80:707). To determine whether CTB-conjugated antigen influenced the phenotype of different APC populations, antigen-pulsed or untreated DC or MØ were incubated for 24 hours in the presence or absence of antigen-specific TCR-transgenic T cells and then analysed for levels of MHC II, CD40, CD80 and CD86 by FACS. Contaminating T cells were gated away using a PerCP-labeled anti-CD3 antibody. When DC had been incubated in the absence of any antigen or any specific T cells, they expressed high levels of MHC II, CD40, CD80 and CD86, whereas MØ incubated in the same way had low expression of MHC II and CD40 and high levels of CD80 and CD86 (not shown).

Incubation of antigen-pulsed APC together with OVA-specific TCR-transgenic T-cells induced a phenotypic change in the APC, as shown for MØ. MØ pulsed with free OVA or OVA peptide had significantly enhanced surface expression of both CD40 and CD86 as compared to un-pulsed MØ (data not shown). The levels of MHC II remained unaltered while a weak up-regulation of CD80 was observed. When APC had been pre-treated with CTB coupled OVA or OVA peptide, the cell surface densities of both CD40 and CD86 were further enhanced. Interestingly, APC that had been pre-treated with CTB-OVA or CTB-OVA peptide up-regulated CD40 and CD86 on their cell surfaces also in the absence of specific T-cells (not shown), although to a much lesser extent than when T-cells were present. Thus, CTB-coupled antigen per se can induce maturation of APC.

CT has previously been shown to enhance the cell-surface levels of the co-stimulatory molecules CD80 and CD86 on DC. We found that, in addition to OVA-CTB, OVA-CT and CT, but not free OVA or free CTB, also enhanced the expression of CD80 and CD86 on the pulsed DC (data not shown).

We further evaluated the cytokine profile associated with the immune response. The levels of IL-1β, IL-12 p40, IL-18, RANTES and MIP-1α were measured in 24 hour culture supernatants from DC pulsed with either free OVA, CTB or CT, or with OVA-CTB and OVA-CT constructs (FIG. 3). We could not detect IL-18 in any of the samples. Similarly, the levels of secreted IL-12 were overall low, even though pulsing the DC with OVA, CTB, OVA-CTB or OVA-CT induced a small increase in IL-12 p40 production. CT alone did not induce any IL-12.

Instead, both native, OVA-CTB, CT and OVA-CT constructs had a more pronounced effect on the production of IL-1β, RANTES and MIP-1α (FIG. 3B-D, lanes 4-6). Thus, pulsing of DC with CT, but not OVA or CTB, induced an IL-1β response. This IL-1β response was even stronger when DC were pulsed with either OVA-CTB or OVA-CT (FIG. 3B, lanes 4 and 6). OVA-CTB also induced DC to produce RANTES, and this production was further enhanced following incubation with OVA-CT, whereas neither native CT, CTB nor OVA had any significant effect (FIG. 3C). CT, OVA-CT and OVA-CTB had a reversed effect on MIP-1α production (FIG. 3D). Thus, untreated DC as well as DC pulsed with either OVA or CTB produced a substantial amount of MIP-1α. This production was completely abrogated by CT or OVA-CT treatment. OVA-CTB could also block MIP-1α production but not as efficiently as CT (FIG. 3D, lane 4).

### Example 7

### Vaccination with DC Pulsed-Treated ex vivo with Tumor-Specific Antigen in Conjunction with CT Induces Antigen-Specific CTL and can Eliminate an Already Established Tumor

Based on the strong immunostimulatory effects on DC and other APC by ex vivo treatment with specific antigen in conjunction with either CTB or CT and the differential steering of the immune response towards Th1 and Th2 by CT and CTB, respectively, we were interested to see if such ex vivo treatment of DC would also empower the DC with capacity to induce an antigen-specific tumor suppressive immune response in mice in vivo. This was tested with the C57/B1 mouse thymoma tumor cell line EG.7 which expresses OVA transgenically including a defined OVA peptide in association with MHC class I thus allowing for OVA peptide specific cytotoxic lymphocytes (CTL) to attack and kill the tumor cells in an antigen-specific manner. When injected intracutaneously into C57/B1 mice, the EG.7 cells form a progressively growing cutaneous tumor the size of which can be easily and no-invasively measured with a caliper.

The EG.7 cells were propagated by tissue culture in Iscove's medium and were divided one day before injection into C57/B1 mice so as to be in a non-confluent state when injected, 2×10⁶ cells intracutaneously into C57/B1 mice. After 3 days when the EG.7 cells have established a small but definitive cutaneous tumor, with a mean diameter of 6 mm, the mice were divided into 5 treatment groups which received intravenous infusion/“vaccination” with differently ex vivo treated DC or treatment with PBS injection only as shown in Table III, a treatment which was repeated 7 days later when the mean tumor diameter in PBS-treated animals had increased to 11 mm. The results are summarized in Table III. In comparison with the PBS treated animals, vaccination with DC did not result in any measurable suppression until day 11, ie., one day after the second DC administration when there was a small but clear reduction in tumor size in the group of mice vaccinated with OVA-CT-pulsed DC. During the next few days the tumors in this latter group of animals practically melted away to be below measurable size in all but one animal on day 16 and undetectable in all animals on day 21 (Table III). In groups given DC treated ex vivo with OVA alone, OVA-CTB conjugate or not treated there was a stagnation in tumor growth from day 10 as compared with the PBS-treated animals but with no differences between these groups and with no evidence of any absolute decrease in tumor size in any group (Table 3). Clearly, the ex vivo treatment with OVA-CT rendered the DC capable of practically eliminating an already established tumor in an antigen-specific manner, a manifestation which was associated with the presence of OVA-specific CTL in spleen from mice within 5 days after 2 vaccinations with OVA-CT pulsed DC as tested with splenic cells on Cr51 labeled EG.7 by classical CTL assay.

TABLE 3

| | Tumor size, mm diameter (mean) | | | | |
|---|---|---|---|---|---|
| Treatment | Day 4 | Day 9 | Day 11 | Day 16 | Day 21 |
| PBS | 7 | 11.5 | 11 | 16 | 20.5 |
| Untreated | 6 | 10.5 | 12 | 11.5 | 13.5 |
| DC | | | | | |
| OVA- | 6 | 9.5 | 10 | 10 | 12.5 |
| pulsed DC | | | | | |
| OVA- | 8 | 10.5 | 10 | 10 | 12 |
| CTB- | | | | | |
| pulsed DC | | | | | |
| OVA-CT- | 7 | 10.5 | 8 | 1** | 0** |
| pulsed DC | | | | | |

**signifies a p value of <0.01 in comparison with all other treatment groups

### Example 10

### Treatment of Human DC and Other Human APC with Antigen in Conjunction with CT or CTB Increases Antigen-Specific T Cell Responses

DC were generated from PBMC of two persons pre-tested to react with TT in standard T cell proliferation tests in vitro. These DC were pulse-treated with TT in conjunction with CT or CTB or not treated as specified in Table 4 before added to and incubated with autologous T cells in a proliferation test. The results are shown in Table IV and show that both CTB, marginally when only mixed with TT but strongly when conjugated with TT, and especially CT, whether only mixed with or linked to TT, increased the TT-specific T cell proliferative response in much the same way as previously described for the effects of another protein antigen (OVA) in conjunction with CTB or CT on mouse DC in similar tests with mouse T cells, see above.

TABLE 4

| | T cell response, | T cell response, |
|---|---|---|
| | individual 1, | individual 2, |
| | cpm (mean | cpm (mean |
| APC | of triplicates) | of triplicates) |
| DC | 140 | 380 |
| DC pulsed with TT | 2,100 | 4,400 |
| DC pulsed with TT + CTB | 3,000 | 6,200 |
| 0.1 mcg/ml | | |
| DC pulsed with TT + CT | 17,000 | 17,700 |
| 0.1 mcg/ml | | |
| DC pulsed with TT + CT | 8,000 | 15,500 |
| 0.001 mcg/ml | | |
| DC pulsed with TT − CTB | 11,000 | 13,600 |
| conjugate | | |
| DC pulsed with TT − CT | 22,000 | 29,800 |
| conjugate | | |

Similar results were obtained when human total APC instead of DC were prepared and treated with these different formulations of TT in conjunction with CT or CTB and used for stimulation of T cell proliferative responses: again treatment of the APC with TT-CTB, TT-CT or TT+CT increased the antigen-specific T cell proliferative response much over that obtained with APC pulsed with TT alone although to a lesser extent (about 5-fold lower cpm values) than the responses obtained with the correspondingly treated DC.

Discussion

We have investigated the immunomodulating capacity of CT and CTB on DC vaccination. Prior to injection into syngeneic mice, bone marrow-derived DC were pulsed with either free OVA, OVA linked to CT or CTB, or mixtures of OVA and CT or CTB. The subsequent immune responses were measured after two DC vaccinations and then again after a subsequent OVA challenge. We found that CTB is an efficient carrier molecule for ex-vivo antigen-pulsing of DC, strongly promoting Th2 responses. Treatment of DC with CT adjuvant also markedly enhanced their immunostimulatory capacity but instead predisposed for Th1 responses thus giving rise to a mixed Th1/Th2 response. The apparently different carrier and adjuvant functions of CT and CTB could be combined for maximal potency if the antigen was linked directly to CT prior to DC pulsing, and then predisposed even stronger for a Th1 response and also induced an antigen-specific CTL response. The Th1 inducing capacity of CT and OVA-CT could be linked to a strong suppression of MIP-1α production by the pulsed DC combined with an upregulation of RANTES production.

Our finding that CTB was an efficient carrier protein for DC vaccination in vivo corroborates our previous in vitro findings. In these studies coupling of antigen to CTB increased the efficacy of antigen presentation by DC and other APC and dramatically lowered the dose of antigen required for efficient presentation, effects that could be linked to an increased uptake of the coupled antigen via binding to the GM1 receptor and up-regulated expression of CD40 and CD86 on the APC (George-Chandy et al., Infect. Immun. 2001; 69:5716. The responses obtained in this study following vaccination with OVA-CTB-pulsed DC were Th2 biased (high titers of OVA-specific IgG1 but no IgG2a) which most likely reflects the fact that CTB-treatment of DC did not or only marginally induce the production of Th1-promoting cytokines such as IL-12 or IL-18. Instead, CD40, which is upregulated on OVA-CTB-treated DC following interaction with T-cells (George-Chandy et al., Infect. Immun. 2001; 69:5716), has recently been shown to induce Th2 development (MacDonald et al., J. Immunol. 2000; 168:537). Furthermore, these results are in line with in vivo data showing that orally administered CTB-coupled antigen can be used as a treatment to deviate the immune response from Th1-mediated pathology (Sun et al., Proc. Natl. Acad. Sci. USA 1996; 93:7196, Tarkowski et al. Artritis and Rheumatism 1999; 42:1628, Sun et al. Int Immunol. 2000; 19:1449).

We further show that CT was a strong adjuvant for DC vaccination enhancing both T-cell and B-cell responses. CT has previously been shown to enhance the cell-surface expression of both MHC, co-stimulatory molecules and chemokine receptors on DC and to affect their secretion of cytokines such as IL-12 and TNF-α (Gagliardi et al., Eur J Immunol 2000;30:2394). We also found a strong up-regulation of co-stimulatory molecules, in particular CD80. However, contrary to previous reports (Gagliardi et al., Eur J Immunol 2000;30:2394), we found that CT-per se induced the secretion of IL-1β from DC. Similar observations have been made with related cell types including macrophages (Cong et al. J. Immunol. 1997; 159:5301, Bromander et al., J. Immunol. 1991; 146:2908, Foss et al. Infect. Immunol. 1999; 67:5275). IL-1 not only induces the maturation of DC (Sallusto et al., J Exp Med 1995; 182:389), but is also an efficient mucosal adjuvant when co-administered with protein antigens (Staats et al. J. Immunol. 1999; 162:6141) and might be responsible for a substantial part of CT's adjuvant activity (Bromander et al., J. Immunol. 1991; 146:2908, Foss et al. Infect. Immunol. 1999; 67:5275, Staats et al. J. Immunol. 1999; 162:6141).

Coupling of antigen directly to CT was superior to pulsing DC with OVA-CTB in the presence of CT which could reflect the joint targeting of antigen and adjuvant to the same DC or possibly to the same sub-cellular compartment. The alternative possibility that there is a competition between CT and OVA-CTB for GM1 binding which leads to a lower uptake of OVA in DC pulsed with OVA-CTB plus CT compared to DC pulsed with OVA-CT is less likely inasmuch as other in vitro studies have shown that such competition would require >100-fold higher concentrations of CT than used in the present study (Jan Holmgren, unpublished observations).

The adjuvant action of CT drove the immune response strongly towards a Th1 type response. This occurred despite a lack of IL-12 or IL-18 priming, and could not be attributed to IL-1β production (as OVA-CTB and OVA-induced comparable levels of IL-1β). Instead, we found that OVA-CT influenced the secretion of chemokines from DC to a larger extent than did OVA-CTB. In untreated DC, the w/w ratio of RANTES to MIP-1α was 0.6. Following OVA-CTB treatment, this ratio increased to 34, and after treatment with OVA-CT to 172. Both RANTES and MIP-1α have a strong impact on Th1/Th2 development. Inclusion of the gene for RANTES into a DNA vaccine promoted Th1 responses (increased IgG2a/IgG1 ratio and IFN-garmma responses as well a stronger induction of cytotoxic CD8+ T-cells) and enhanced the protection against a viral infection. When the gene for MIP-1α was similarly included into DNA vaccines, the opposite effect was achieved, i.e. a strong Th2 response with enhanced antibody production, decreased IgG2a/IgG1 ratios and a decreased protective capacity against a viral infection (Sin et al., J. Virol 2000; 74:11173, Kim et al., J Interferon Cytokine Res 2000; 20:487). Furthermore, RANTES has been associated with Th1 responses and MIP-1α with Th2 responses in mice infected with Schistosoma mansoni (Park et al., Infect. Immun. 2001; 69:6755). Thus, it appears that CT promotes Th1 development by affecting RANTES production positively and MIP-1α production negatively, in an IL-12 independent fashion.

However, other factors could also influence the Th1/Th2 balance. CT-pulsed DC carried considerable quantities of CT adjuvant on their cell surfaces. We do not know to what extent such cell-surface bound CT retains adjuvant properties towards adjacent cells, and if cell-surface bound CT would potentiate Th1 development. However, our preliminary in vitro data show that the toxic effects of CT on T-cells are lost if CT is attached to DC, suggesting that DC cell-surface bound CT does not enter adjacent cells. It is also debated whether free CT would induce a Th1 and/or a Th2 response. Several studies have shown that CT per se primes for an exclusive Th2 responses by inhibiting IL-12 production (Braun et al., J. Exp. Med. 1999; 189:541, Gagliardi et al., Eur. J. Immunol. 2000; 30:2394). However, when CT is administered mucosally as an adjuvant it induces a combined Th1/Th2 response (Hörnquist et al., Eu.r J. Immunol 1993; 23:2136, Kjerrulf et al., Immunol. 1997; 92:60) in a similar fashion to our CT-pulsed DC and, opposite to CTB, it aggravates Th1 mediated DTH and autoimmune tissue reactions (Sun et al., Proc. Natl. Acad. Sci. USA 1996; 93:7196) Another possibility is that the levels of co-stimulatory molecules on the injected CT-pulsed DC affect Th1/Th2 commitment. CT-treated or OVA-CT-pulsed DC upregulated their expression of CD80 to a much higher degree than OVA-CTB-pulsed DC. Even though no consensus has been reached regarding the roles of CD80 and CD86 in Th1/Th2 development, there are several reports showing that high levels of CD80 does drive Th1 commitment, either through a Th1-specific pathway or simply by offering more co-stimulation (Kuchroo et al., Cell 1995; 80:707, Schweitzeretal., J. Immunol. 1997; 158:2713, Soos et al., lnt. Immunol. 1999; 11:1169). Furthermore, we can neither rule out the possibility that CT and CTB affect intracellular processing and loading of OVA onto MHC molecules in different ways, nor that chemokine receptor expression and intracellular survival of OVA-CTB- and OVA-CT-pulsed DC might differ. Ex vivo treatment of DC with CT-conjugated antigen followed by infusion of the treated DC into mice also induced antigen-specific CTL and could eliminate even an already established tumor in an antigen-specific way. Irrespective of the underlying mechanism(s), the use of CT as a DC adjuvant should be especially interesting for DC vaccination against selected virus infections or tumors, where a strong Th1 and CTL response is desired. The normal toxicity of CT which precludes its use as an adjuvant for in vivo administered vaccines would appear to be irrelevant for DC vaccination, where the CT treatment takes place in vitro with extensive washing of DC before they are infused in vivo.

It was interesting to note that chemical conjugates of OVA-CTB and OVA-CT had a stronger impact on cytokine and chemokine production than any of the native proteins alone, including CT. Thus, the highest levels of IL-12, IL-1β and RANTES produced by the DC were induced by the chemical conjugates. CT, OVA-CT and OVA-CTB, but not free OVA or free CTB, also induced an enhanced expression of CD80 and CD86. We do not know why chemical conjugates have such a strong immunomodulatory capacity on the production of cytokines and chemokines and on the levels of co-stimulatory molecules, but believe that it is related to the linked binding and uptake of CT/CTB and specific antigen rather than to any chemical modifications induced by the coupling procedure. Irrespective of the underlying mechanism, this strong effect on DC phenotype and on cytokine and chemokine production could explain part of the immunopotentiating capacity of CT and CTB conjugates.

In summary, both CT and CTB can be utilized to enhance the responsiveness to DC vaccination. Conjugating antigen to CT or CTB prior to DC pulsing could enhance the subsequent immune response through several mechanisms; (i) an enhanced uptake and presentation of the conjugated protein, (ii) IL-1β and, especially using CT, RANTES co-stimulation, and (iii) enhanced expression of co-stimulatory molecules. More importantly, through the selective use of either CT or CTB as carrier antigens one can direct the immune response towards either Th1 and CTL or Th2 responses, respectively, in a process that appears to be associated with chemokine expression and levels of co-stimulatory molecules.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

It is further to be understood that all values are approximate, and are provided for description.

Patents, patent applications, publications, product descriptions, and protocols are cited throughout this application, the disclosures of which are incorporated herein by reference in their entireties for all purposes.

1. A method for inducing an immune response directed against a tumor cell in a mammal comprising the steps of:
a) contacting antigen presenting cells (APCs) ex vivo with
i) cholera toxin, its B subunit or a related toxin or cell-binding protein
ii) an antigen specific for said tumor cell at a concentration and for a time effective to activate said APCs and promote an immune response directed against said tumor cell;
b) washing and removing any unbound amount of said cholera toxin or its B subunit and said tumor antigen from said APCs; and
c) administering in vivo to said mammal, an amount of said APCs effective to induce an immune response against said tumor cells.

2. The method of claim 1 wherein the related toxin or cell binding protein is selected from the group consisting of ADP-ribosylating toxins, their subunits, plant lectins, and viral attachment proteins.

3. The method of claim 1 wherein said tumor cell is selected from lung, colorectal, pancreatic, prostate, ovarian, breast, multiple myeloma, leukemia and melanoma tumors.

4. The method of claim 1 wherein said tumor is a solid tumor.

5. The method of claim 1 wherein said tumor is a disseminated tumor.

6. The method of claim 1 wherein said tumor is a metastatic tumor.

7. The method of claim 1 wherein said cholera toxin or B subunit or related protein is chemically coupled to said tumor antigen.

8. The method of claim 1 wherein said cholera toxin or its B subunit is genetically coupled to said tumor antigen.

9. The method of claim 1 wherein said tumor antigen comprises an extract from said tumor, wherein said tumor extract has been inactivated.

10. The method of claim 9, wherein said extract is a membranous preparation.

11. The method of claim 10, wherein said membranous preparation has been inactivated.

12. The method of claim 1 wherein said APCs are selected from dendritic cells, B cells, macrophages, mast cells and epithelial cells.

13. The method of claim 12 wherein said epithelial cell are selected from keratinocytes, buccal epithelial, gastro-intestinal epithelial and genital tract epithelial cells.

14. The method of claim 1 wherein said APCs are isolated from the same mammalian host to whom said APCs will be administered.

15. The method of claim 1 wherein said APCs are isolated from a mammalian host different from the one to whom said APCs will be administered.

16. The method of claim 1 wherein said cholera toxin or related protein is mixed with said tumor antigen.

17. The method of claim 1 wherein an amount of cholera toxin effective to increase said immune response is added to said contacting step.

18. An isolated APC treated according to the method of claim 1.

19. The APC of claim 18 wherein said APC is selected from dendritic cells, B cells, macrophages, mast cells and epithelial cells.

20. The APC of claim 19 wherein said epithelial cells are selected from the group consisting of keratinocytes, buccal epithelial, gastrointestinal epithelial and genital tract epithelial cells.

21. A method for inducing an immune response against an antigen in a mammal comprising the steps of:
a) contacting antigen presenting cells (APCs) ex vivo with
i) cholera toxin, its B subunit or related toxin or cell-binding protein
ii) a non-self antigen or group of antigens at a concentration and for a time effective to activate said APCs to promote an immune response directed against said antigen or antigens;
b) washing and removing said unbound cholera toxin or B subunit and non-self antigen or antigens from said APCs; and
c) administering to said mammal, an amount of said APCs effective to induce an immune response against said antigen or antigens.

22. The method of claim 21 wherein said toxin is a cell-binding protein.

23. The method of claim 21, wherein said cell-binding protein is selected from ADP-ribosylating toxins, their subunits, plant lectins, and viral attachment proteins.

24. The method of claim 21, wherein said non-self antigen is derived from a bacterium, a virus, a fungus, a protozoan or, a helminth.

25. The method of claim 21, wherein said non-self antigen is a protein, peptide, carbohydrate, lipid or complex thereof.

26. The method of claim 21, wherein said non-self antigen is a DNA encoding a non-self protein, peptide, carbohydrate, lipid or complex thereof.

27. The method of claim 21, wherein said cholera toxin or B subunit is chemically coupled to said immunogenic peptide or protein.

28. The method of claim 21 wherein said cholera toxin B subunit is genetically coupled to said immunogenic peptide or protein.

29. The method of claim 21, wherein said APCs are selected from dendritic cells, B cells, macrophagaes, mast cells and epithelial cells.

30. The method of claim 28, wherein said epithelial cell are selected from keratinocytes, buccal epithelial, gastro-intestinal epithelial and genital tract epithelial cells.

31. The method of claim 21, wherein said APCs are isolated from the same mammalian host to whom said APCs will be administered.

32. The method of claim 21, wherein said APCs are isolated from a mammalian host different from the one to whom said APCs will be administered.

33. The method of claim 21, wherein said cholera toxin or B subunit is mixed with said immunogenic peptide or protein.

34. The method of claim 21, wherein an amount of cholera toxin effective to increase said immune response is added to said contacting step.

35. An isolated antigen-presenting cell (APC) treated according to the method of claim 21.

36. The APCs of claim 35, wherein said APC is selected from dendritic cells, B cells, macrophages, mast cells and epithelial cells.

37. The APC of claim 36 wherein said epithelial cell are selected from the group consisting of keratinocytes, buccal epithelial, gastro-intestinal epithelial and genital tract epithelial cells.
